(19) United States
(12) Patent Application Publication
(10) Pub. No.: US 2023/0140044 A1
(43) Pub. Date: May 4, 2023

(54) MICRORNAS FOR THE PREVENTION OF CLINICAL VENOUS THROMBOEMBOLIC DISEASE

(71) Applicant: UNIVERSITY OF MASSACHUSETTS, Boston, MA (US)

(72) Inventors: Cahan Mitchell, Holden, MA (US); Basadonna Giacomo, Haddam, CT (US); Freedman Jane, Boston, MA (US); Tanriverdi Kahraman, Northborough, MA (US); Kucukural Alper, Cambridge, MA (US)

(21) Appl. No.: 17/913,100

(22) PCT Filed: Mar. 19, 2021
(86) PCT No.: PCT/US21/23156

§ 371 Date: Sep. 20, 2022

Related U.S. Application Data
(60) Provisional application No. 62/992,696, filed on Mar. 20, 2020.

(51) Int. Cl. C12N 15/113

(52) U.S. Cl. CPC C12N 15/113; C12N 2310/113; C12N 2310/3519; C12N 2310/51

(57) ABSTRACT

The present invention is related to the field of cardiovascular thrombosis. In particular, compositions and methods that modulate gene regulation of cardiovascular thrombosis. For example, such gene regulation may be controlled by modulating microRNA binding. The present invention contemplates inhibitor compounds, such as antisense inhibitors, that compete with miRNA binding to specific gene regulatory sites on the SERPINC1 gene.

### FIELD OF THE INVENTION

The present invention is related to the field of cardiovascular thrombosis. In particular, compositions and methods that modulate gene regulation of cardiovascular thrombosis. For example, such gene regulation may be controlled by modulating microRNA binding. The present invention contemplates inhibitor compounds (e.g., antisense inhibitors) that compete with miRNA binding to specific gene regulatory sites on the SERPINC1 gene.

### BACKGROUND

Venous thromboembolism (VTE) is often associated with prolonged periods of immobility or following surgery.¹ In the United States, VTE, which includes deep venous thrombosis (DVT) and pulmonary embolism (PE), is a major cause of morbidity and mortality affecting approximately 900,000 patients annually and resulting in 300,000 deaths from PE per year.² Rates of thrombotic events in general surgery patients range from 15-30% for DVT and 0.2-0.9% for PE among those who have not received prophylaxis, compared to 9% and 0.3% in patients who have received low molecular weight heparin.¹ Although the risk of DVT and PE is significantly reduced with primary prophylaxis, VTE from all causes continues to represent a significant health care burden, accounting for more than half a million hospitalizations in the US per year and annual estimated treatment costs between 7-12 billion USD.^{1,3,4} Interestingly, certain hibernating animals, such as the American black bear (Ursus americanus), do not appear to suffer from VTE or its sequelae, despite experiencing extended periods of immobility.^{1,5}

What is needed in the art are therapeutics and methods of treatments that target gene regulation of venous thrombotic disorders.

### SUMMARY OF THE INVENTION

The present invention is related to the field of cardiovascular thrombosis. In particular, compositions and methods that modulate gene regulation of cardiovascular thrombosis. For example, such gene regulation may be controlled by modulating microRNA binding. The present invention contemplates inhibitor compounds that compete with miRNA binding to specific gene regulatory sites on the SERPINC1 gene.

In one embodiment, the present invention contemplates a composition comprising an anti-miR oligonucleotide (e.g., an antisense inhibitor). In one embodiment, the antisense inhibitor comprises a plurality of nucleic acids conjugated by a phosphorothioate linker. In one embodiment, at least one of the plurality of nucleic acids comprises a chemical modification. In one embodiment, the chemical modification includes, but is not limited to, a 2′-OMe modification, a locked nucleic acid (LNA) modification or a 2′-deoxy modification. In one embodiment, the 3′ end of the antisense inhibitor comprises a GalNAc conjugate. In one embodiment, the antisense inhibitor includes, but is not limited to, anti200bc, anti200a+141, anti429, anti18a and/or anti19b, as described herein.

In one embodiment, the present invention contemplates a method, comprising: a) providing; i) a patient exhibiting at least one symptom of a cardiovascular thrombosis and comprising a serpine family C member 1 gene (SERPINC1), said SERPINC1 gene having at least one micro-ribonucleic acid (miRNA) binding site; ii) an inhibitory compound having affinity for said at least one miRNA binding site; b) administering said inhibitory compound such that said at least one symptom is reduced. In one embodiment, the at least one miRNA binding site is selected from the group consisting of an miR-141-3p binding site, an miR-200a-3p binding site, an miR-200b-5p binding site, and an miR-200c-3p binding site. In one embodiment, the inhibitory compound includes, but is not limited to, anti200bc, anti200a+141, anti429, anti18a and/or anti19b, as described herein. In one embodiment, the administering further comprises increasing expression of antithrombin messenger ribonucleic acid (mRNA) from said SERPINC1 gene. In one embodiment, the increased antithrombin RNA increases translation of an antithrombin protein. In one embodiment, the administering further comprises preventing development of said cardiovascular thrombosis. In one embodiment, the administering further comprises treating a developed cardiovascular thrombosis. In one embodiment, the inhibitory compound is a nucleic acid sequence. In one embodiment, the inhibitory compound is an antibody. In one embodiment, the antibody is a polyclonal antibody. In one embodiment, the antibody is a monoclonal antibody.

In one embodiment, the present invention contemplates a method of manufacture of an antisense inhibitor (e.g., an anti-miR oligonucleotide) comprising; providing a) an automated RNA synthesizer on a 1 μmol GalNAc (TEG)CPG support; and ii) modification compounds including, but not limited to 2′-OMe and locked nucleic acid (LNA) phosphoramidites; b) synthesizing a plurality of nucleic acids according to a standard RNA phosphoramidite synthesis cycle, comprising (i) detritylation, (ii) coupling, (iii) capping, and (iv) iodine oxidation to phosphate or sulfurization; c) coupling the phosphoramidites with 2-cyanoethyl phosphoramidite to form an oligonucleotide; d) deprotecting and cleaving the oligonucleotide from the with 40% Methylamine-28% NH4OH (1:1, v/v) for 2 h at ambient temperature for deprotection and cleavage of oligonucleotides from the GalNAc (TEG)CPG support. In one embodiment, the method further comprises purifying the oligonucleotides by anion exchange HPLC. In one embodiment, the method further comprises desalting the purified oligonucleotides by Sephadex G-25. In one embodiment, the method further comprises characterizing the oligonucleotides by electrospray ionization mass spectrometry (ESI-MS) analysis.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

The term “VTE” refers to any condition related to a venous thromboembolism.

The term “DVT” refers to any condition related a deep vein thrombosis.

The term “PE” refers to any condition related to a pulmonary embolism.

The term “AT” refers to ant antithrombin protein having anti-coagulant activity.

The term “SERPINC1” refers to a gene encoding a serpin family C member 1 protein (e.g., antithrombin III).

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, and it can be expressed as the ratio LD50/ED50. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “inhibitory compound” as used herein, refers to any compound capable of interacting with (i.e., for example, attaching, binding etc.) to a binding partner under conditions such that the binding partner becomes unresponsive to its natural ligands. Inhibitory compounds may include, but are not limited to, small organic molecules, nucleic acid sequences, antibodies, and proteins/peptides.

The term “small organic molecule” as used herein, refers to any molecule of a size comparable to those organic molecules generally used in pharmaceuticals. The term excludes biological macromolecules (e.g., proteins, nucleic acids, etc.). Preferred small organic molecules range in size from approximately 10 Da up to about 5000 Da, more preferably up to 2000 Da, and most preferably up to about 1000 Da.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, etc.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term, “antithrombins” or “antithrombin drug” as used herein, refers to any drug that inhibits or reduces thrombi formation and include, but are not limited to, bivalirudin, ximelagatran, hirudin, hirulog, argatroban, inogatran, efegatran, or thrombomodulin.

The term, “anticoagulants” or “anticoagulant drug” as used herein, refers to any drug that inhibits the blood coagulation cascade. A typical anticoagulant comprises heparin, including but not limited to, low molecular weight heparin (LMWH) or unfractionated heparin (UFH). Other anticoagulants include, but are not limited to, tinzaparin, certoparin, parnaparin, nadroparin, ardeparin, enoxaparin, reviparin or dalteparin. Specific inhibitors of the blood coagulation cascade include, but are not limited to, Factor Xa (FXa) inhibitors (i.e., for example, fondaparinux), Factor IXa (FIXa) inhibitors, Factor XIIIa (FXIIIa) inhibitors, and Factor VIIa (FVIIa) inhibitors. Further, a protein such as antithrombin may also act as an anticoagulant. The antithrombin protein is encoded by the SERPINC1 gene whose expression is regulated by numerous miRNAs as described herein.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term, “purified” or “isolated”, as used herein, may refer to any compound that has been subjected to treatment (i.e., for example, fractionation) to remove various other components, and which composition substantially retains its expressed biological activity. Where the term “substantially purified” is used, this designation will refer to any compound which forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to ‘apparent homogeneity” such that there is single compound (i.e., for example, based upon SDS-PAGE or HPLC analysis). A purified composition is not intended to mean that all trace impurities have been removed.

As used herein, the term “substantially purified” refers to any compound that is removed from their natural environment, isolated or separated, and are at least 60% free, preferably 75% free, and more preferably 90% free from other components with which they are naturally associated. An “isolated compound” is therefore a substantially purified compound.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The term “portion” when used in reference to a nucleotide sequence refers to fragments of that nucleotide sequence. The fragments may range in size from 5 nucleotide residues to the entire nucleotide sequence minus one nucleic acid residue.

As used herein, the term “antisense” is used in reference to RNA sequences which are complementary to a specific RNA sequence (e.g., mRNA). Antisense RNA may be produced by any method, including synthesis by splicing the gene(s) of interest in a reverse orientation to a viral promoter which permits the synthesis of a coding strand. Once introduced into a cell, this transcribed strand combines with natural mRNA produced by the cell to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

As used herein, the terms “siRNA” refers to either small interfering RNA, short interfering RNA, or silencing RNA. Generally, siRNA comprises a class of double-stranded RNA molecules, approximately 20-25 nucleotides in length. Most notably, siRNA is involved in RNA interference (RNAi) pathways and/or RNAi-related pathways. wherein the compounds interfere with gene expression.

As used herein, the term “shRNA” refers to any small hairpin RNA or short hairpin RNA. Although it is not necessary to understand the mechanism of an invention, it is believed that any sequence of RNA that makes a tight hairpin turn can be used to silence gene expression via RNA interference. Typically, shRNA uses a vector stably introduced into a cell genome and is constitutively expressed by a compatible promoter. The shRNA hairpin structure may also cleaved into siRNA, which may then become bound to the RNA-induced silencing complex (RISC). This complex binds to and cleaves mRNAs which match the siRNA that is bound to it. As used herein, the term “microRNA”, “miRNA”, or “pRNA” refers to any single-stranded RNA molecules of approximately 21-23 nucleotides in length, which regulate gene expression. miRNAs may be encoded by genes from whose DNA they are transcribed but miRNAs are not translated into protein (i.e. they are non-coding RNAs). Each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency.

This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding the probe will not hybridize to the second non-complementary target.

The terms “homology” and “homologous” as used herein in reference to amino acid sequences refer to the degree of identity of the primary structure between two amino acid sequences. Such a degree of identity may be directed a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity. An oligonucleotide sequence which is a “homolog” is defined herein as an oligonucleotide sequence which exhibits greater than or equal to 50% identity to a sequence, when sequences having a length of 100 bp or larger are compared.

As used herein, the term “hybridization” is used in reference to the pairing of complementary nucleic acids using any process by which a strand of nucleic acid joins with a complementary strand through base pairing to form a hybridization complex. Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids.

As used herein the term “hybridization complex” refers to a complex formed between two nucleic acid sequences by virtue of the formation of hydrogen bounds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., C0 t or R0 t analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH (fluorescent in situ hybridization)).

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region. As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

As used herein, the term “structural gene” refers to a DNA sequence coding for RNA or a protein. In contrast, “regulatory genes” are structural genes which encode products which control the expression of other genes (e.g., transcription factors).

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “bind” as used herein, includes any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measuring. The “binding” interaction may be brief as in the situation where binding causes a chemical reaction to occur. That is typical when the binding component is an enzyme and the analyte is a substrate for the enzyme. Reactions resulting from contact between the binding agent and the analyte are also within the definition of binding for the purposes of the present invention.

The term “binding site” as used herein, refers to any molecular arrangement having a specific tertiary and/or quaternary structure that undergoes a physical attachment or close association with a binding component. For example, the molecular arrangement may comprise a sequence of amino acids. Alternatively, the molecular arrangement may comprise a sequence a nucleic acids. Furthermore, the molecular arrangement may comprise a lipid bilayer or other biological material.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1 presents exemplary data showing eighty-nine (89) differentially expressed miRNAs in bear plasma. Forty (40) miRNAs were observed to be statistically significantly upregulated in hibernating black bears (red dots: n=10;). Forty-nine (49) miRNAs were observed to be statistically significantly upregulated in active black bears (blue dots; n=11).

FIG. 2 presents exemplary data showing that miRNAs miR-141-3p, miR200a-3p, miR-200b-5p, and miR-200c-3p associated with kidney SERPINC1 are differentially expressed in plasma of hibernating black bears (red bar: n=10) and active black bears (blue bar: n=11).

FIG. 3 presents an illustration showing how MiRNAs regulate a hemostatic system. The depicted pathways demonstrate that increased levels of circulating antithrombin (AT) can result from downregulated levels of miR-141-3p, miR-200a-3p, miR-200b-5p, and miR-200c-3p. This may explain the observed increased expression of the SERPINC1 gene in hibernating American black bears.

FIG. 4 presents an exemplary miRNA expression construct.
FIG. 4A: A representative nucleic acid sequence for a double stranded plasmid comprising a pol II promoter sequence, an miR220b antisense molecule sequence, an SV40_late_16s_int sequence and a BGHpA sequence.
FIG. 4B: A representative schematic of the plasmid of FIG. 4A packaged into an associated adenovirus 8 (AAV8) vector.

FIG. 5 presents exemplary data showing SERPINC1 mRNA expression following the transfection of an antisense nucleic acid with Lipofectamine 3000 as a transfection reagent.
FIG. 5A: Transfection of miR200b into HuH7.5 cells.
FIG. 5B: Transfection of miR200b into HepG2 cells.

FIG. 6 presents exemplary data showing SERPINC1 mRNA expression following the transfection of the miR200b antisense nucleic acid into HuH7.5 cells HuH7.5 cells using the highest concentration of Lipofectamine 3000 in FIG. 5A and 1 μg of DNA for 48 hours.

FIG. 7 presents exemplary data showing SERPINC1 mRNA expression following the transfection of five (5) antisense inhibitors with the RNAimax transfection reagent for 48 hours in HuH.7.5 cells.

FIG. 8 presents exemplary data showing SERPINC1 mRNA expression following the transfection of five (5) antisense inhibitors with GenMute or Lipofectamine 3000 as a transfection reagent for 48 hours in HuH.7.5 cells.

FIG. 9 illustrates several embodiments of chemical modifications to anti-mR oligonucleotides (antisense inhibitors).
FIG. 9A: Nucleotide modifications.
FIG. 9B: 3′ conjugate modification.

FIGS. 10A-E present exemplary LS-mass spectroscopy data of the five (5) antisense inhibitors presented in Table 4.

### DETAILED DESCRIPTION OF THE INVENTION

The present invention is related to the field of cardiovascular thrombosis. In particular, compositions and methods that modulate gene regulation of cardiovascular thrombosis. For example, such gene regulation may be controlled by modulating microRNA binding. The present invention contemplates inhibitory compounds that compete with miRNA binding to specific gene regulatory sites on the SERPINC1 gene.

It has previously been demonstrated that the hibernating bears have significantly different clotting parameters than their active counterparts^{1,5,11,37}. The data presented herein discloses a previously unknown role of miRNAs in the genetic modulation of hemostasis in hibernating bears. For example, at least four miRNAs (e.g., miR-141-3p, miR-200a-3p, miR-200b-5p, and miR-200c-3p) were identified that were differentially expressed in the plasma of American black bears and target the AntiThrombin (AT) gene and/or a Serpin Family C Member 1 (SERPINC1) gene (alternatively known as antithrombin III). MiR-141-3p, miR-200a-3p, miR-200b-5p, and miR-200c-3p are all observed to be significantly downregulated in a winter hibernation state and upregulated in a summer active state. These data suggest that lower miRNA levels would result in higher AT levels in hibernating bears. Consequently, the higher AT levels would provide thrombosis protection during prolonged periods of immobility experienced during hibernation.

In one embodiment, the present invention contemplates compositions and methods related to miRNA regulation of AT and/or SERPINC1 that may provide an opportunity for tailored therapies for thrombotic events (e.g., DVTs and/or PEs) with improved efficacy or more favorable risk profile. These findings also provide information to provide novel therapeutics, for example, through the application of specific miRNA target site inhibitors (e.g., target binding sites), to prevent or treat VTE in humans by inhibiting miRNA hybridization with either the AT gene or the SERPINC1 gene.

### I. Relationships Between Mammalian Hibernation and an Anti-Coagulation State

Hibernating American black bears have significantly different clotting parameters than their summer active counterparts, affording them protection against venous thromboembolism (VTE) during prolonged periods of immobility. It was sought to evaluate if significant differences exist between the expression of miRNAs in the plasma of hibernating black bears compared to their summer active counter parts, potentially contributing to differences in hemostasis during hibernation. MicroRNA sequencing was assessed in plasma from 21 American black bears in summer active (n=11) and hibernating states (n=10) and miRNA signatures during hibernating and active state were established using both bear and human genome. MiRNA targets were predicted using messenger RNA (mRNA) transcripts from black bear kidney cells. Using the bear genome, fifteen (15 miRNAs) were identified as differentially expressed in the plasma of hibernating black bears. Of these miRNAs, four were significantly downregulated and (miR-141-3p, miR-200a-3p, miR-200b-5p, and miR-200c-3p) were predicted to target SERPINC1, the gene for antithrombin (AT), a protein previously shown to be altered in hibernating bears. These findings suggest that hibernating black bears ability to maintain hemostasis and achieve protection from VTE during prolonged periods of immobility may be due to changes in miRNA signatures and possible upregulation of AT expression.

Numerous physiologic changes occur during the cyclical state of hibernation including, but not limited to, lower metabolism, lower core body temperature, lower heart rate and lower respiratory rate.^{6,7} These adaptive changes are believed to result from differential expression of ubiquitous mammalian genes, rather than from the presence of novel genes specific to hibernating animals.′ Recent studies have sought out to detect differential expression of genes involved in the above described physiologic changes observed during hibernation, but few reports evaluate any genetic/epigenetic basis of hemostasis in hibernating mammals.^{7,8}

The American black bear (Ursus americanus) is a well known model for translational research to study evolutionary adaptations during hibernation. Compared to other hibernating species, its weight is most similar to humans (30-200 kilograms) and the black bear maintains higher core body temperature during hibernation (30-36 degrees Celsius) as compared to brown bears (Ursus arctos), hamsters, ground squirrels, and marmots.^{1,7-10} The black bears of North America may remain in hibernation for approximately four to seven months. Most hibernating mammal do not eat, drink, urinate, defecate and elicit very little movement to allow a reduction in metabolic rate by up to 50%^{7,8,11}

It was recently reported that there are distinct differences in hemostatic parameters of the American black bear during hibernation compared to during their active summer months.¹ For example, hibernating bears had markedly prolonged prothrombin time (PT), activated prothrombin time (aPTT), and Kaolin clotting time (KCT) compared to their summer active counterparts. Clotting factor XI and clotting factor XII levels were significantly lower in hibernators, as were levels of antithrombin (AT), fibrinogen, plasminogen, antiplasmin, protein C, and von Willebrand Factor as compared to active black bears.¹

Having established that most coagulation parameters differ significantly between hibernating and summer active black bears, the data presented herein identifies specific microRNA (miRNA) signatures through which these animals may maintain hemostasis despite experiencing numerous physiologic changes that would be expected to favor coagulation. The identification of such anti-coagulation related miRNA signatures provide regulatory targets for novel therapeutics which could be used in the prevention or treatment thrombotic events in humans.

### II. Gene Regulation of Thrombotic Events

The data presented herein show at least four miRNAs that were significantly downregulated in the hibernating black bears (e.g., miR-141-3p, miR-200a-3p, miR-200b-5p, and miR-200c-3p). All of these four miRNAs are known to target the SERPINC1 gene. SERPINC1 is a gene located on chromosome 1q23-35 that is believed responsible for encoding antithrombin (AT), a circulating anticoagulant protein.^{12,15,31-36} AT is believed synthesized in the liver and subsequently released into the blood plasma.³³

AT has been reported to have two functional domains; one active binding site and one binding site for heparin or heparan sulfates present on endothelial cell surfaces.^{12,31-34} Although de novo AT is a weak anticoagulant, AT's anticoagulant efficacy increases 1000-fold when it is bound to heparin sulfate.^{12,31-34} AT primarily effects coagulation by inhibiting clotting factors including, but not limited to, thrombin (factor IIa), factor Xa, factor IXa and/or factor XIa.³¹⁻³⁷ AT has been implicated in various thrombotic disorders that occur as a result of mutations in the SERPINC1 gene.⁴³⁻⁴⁹ More than three hundred (300) mutations in SERPINC1 gene have been identified that may result in either reduced AT production (type I: quantitative defect) or reduced AT activity due to abnormal structure or function (type I: qualitative defect).³¹⁻³⁷ Although inherited AT deficiencies are rare, they are associated with profoundly increased risk of VTE, with an estimated increased risk of approximately 16-fold and a risk of recurrence approximately 4-fold that of the unaffected population.³²

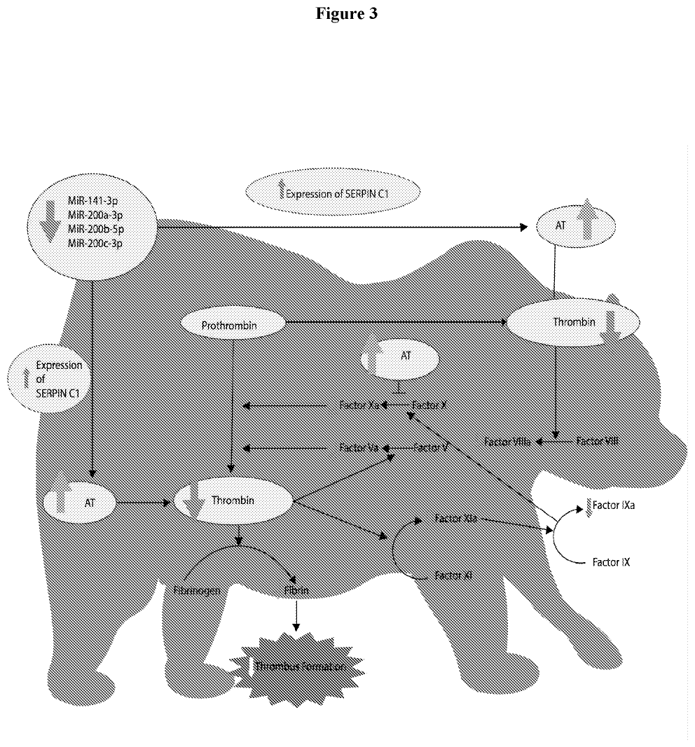

AT levels, along with other coagulation parameters are believed to be significantly different between hibernating and active American black bears. The data presented herein is believed show that AT translation may be modulated through miRNA regulation. MiRNAs generally exert an inhibitory role by preventing translation and/or resulting in destruction of mRNA transcripts.¹² Consequently, a reduced activity of miR-141-3p, miR-200a-3p, miR-200b-5p, and miR-200c-3p would therefore result in an unopposed expression of SERPINC1 gene and increased production of AT. Higher circulating levels of AT would allow for greater inhibition of serine protease factors and reduction in thrombin generating capacity. See, FIG. 3. As previously reported, prolonged aPTT and low factor XI levels have been observed in hibernating black bears.¹ That study also found a significant decrease in circulating AT activity in hibernating versus summer active bears.¹ One explanation for this finding is that during hibernation, the rate of AT degradation and clearance increases through complex formation with activated serine protease factors or vascular wall heparans. The presently observed decrease in miRNA targeting AT during hibernation suggests that upregulation of AT synthesis may be a compensatory regulatory response to support ongoing AT losses.

Other studies that have investigated the role of miRNAs in the regulation of AT. miR-18a and miR-19b expression in the livers of neonatal and adult mice were identified as potential targets for AT and, unlike the presently disclosed data, were found to be inversely correlated with levels of AT mRNA.¹⁴ On the other hand, an anti-sense oligonucleotide that targeted miR19-3b (anti-miR) was studied in an AT regulatory mouse model with the goal of blocking the miRNA-mediated suppression of SERPINC1. The resultant uninhibited SERPINC1 expression resulted in a consistent increase in AT activity.¹² While the study reported that the increase in AT activity was very small, yet statistically significant, the observations support the hypothesis that AT is a regulated anticoagulant and also that the effect of a single miRNA may not entirely explain its regulation.

### III. MicroRNA Regulation

I. Physiology

Previous reports have long speculated regarding a physiological role for microRNAs. For example, plasma microRNAs have been proposed to provide information regarding medical diagnoses and overall therapeutic management. Zhang et al., “Serum/Plasma Micronas And Uses Thereof” United States Patent Application Publication Number 2010/0173288 (herein incorporated by reference). This reference suggests that all detectable microRNAs should be used together in order to evaluate a physiological condition While this reference lists the known presence of a multitude of microRNAs (miR), the reference fails to identify miR-141-3p, miR-200a-3p, miR-200b-5p or miR-200c-3p. Furthermore, the reference fails to disclose that any miR, or combination of miR's could be associated with the regulation of antithrombin.

Differential miR expression has been reported to be useful in the detection of pancreatic disorders. Labourier et al., “Micrornas Differentially Expressed In Pancreatic Diseases And Uses Thereof” United States Patent Application Publication Number 2009/0131348 (herein incorporated by reference). While this reference lists the known presence of a multitude of microRNAs, the reference fails to identify miR-141-3p, miR-200a-3p, miR-200b-5p or miR-200c-3p. Furthermore, the reference fails to disclose that any miR, or combination of miR's could be associated with the regulation of antithrombin.

The potential relevance of miRNAs to cancer detection more generally has also been contemplated. Aharonov et al., “Cancer-Related Nucleic Acids” United States Patent Application Publication Number 2009/0186353 (herein incorporated by reference). The reference lists several thousand potential miRNAs as speculative candidate compounds but provides data for only four miRNAs. The reference fails to identify miR-141-3p, miR-200a-3p, miR-200b-5p or miR-200c-3p. Furthermore, the reference fails to disclose that any miR, or combination of miR's could be associated with the regulation of antithrombin. A global miRNA expression analysis from a large cohort of cancer patients has been reported. Rigoutsos et al., “Leveraging The Presence Or Absence Of miRNA Isoforms For Recommending Therapy In Cancer Patients” WO 2018/076015. In particular, the collected miRNA dataset was programmed for the discrimination between thirty-two (32) different type of cancers listed in The Cancer Genome Atlas. The reference fails to disclose that any miR, or combination of miR's could be associated with the regulation of antithrombin.

Other reports have tried to associate various miRNAs with tissue damage and associated disorders. Sheils, P., “Cellular And Molecular Therapies” WO 2012/020308. In particular, the reference contemplates that the administration of one of several hundred miRNAs might be useful to treat a medical condition related to chronic or acute tissue damage. The reference fails to identify miR-141-3p, miR-200a-3p, miR-200b-5p or miR-200c-3p. Furthermore, the reference fails to disclose that any miR, or combination of miR's could be associated with the regulation of antithrombin.

It has been speculated that miRNAs provide gene expression regulation by interacting with the untranslated region (UTR), usually located at the 3′ end of a specific gene. Nourse et al., “Large-scale identification of functional microRNA targeting reveals cooperative regulation of the hemostatic system” Journal of Thrombosis and Haemostasis, 16:2233-2245 (2018). The reference identifies several miRNAs that interact with the SERPINC1 gene UTR but fails to identify miR-141-3p, miR-200a-3p, miR-200b-5p or miR-200c-3p.

Deep vein thrombosis has been associated with the upregulation of miR-126. Meng et al., “Upregulation of MicroRNA-126 Contributes to Endothelial Progenitor Cell function in deep vein thrombosis via Its Target PIK3R2” J. Cell Biochem. 116:1613-1623 ((2015). The proposed mechanism of this action is to recruit endothelial progenitor cells by suppressing PIK3R2 expression to resolve the thrombi. The reference fails to identify miR-141-3p, miR-200a-3p, miR-200b-5p or miR-200c-3p. Furthermore, the reference fails to disclose that any miR, or combination of miR's could be associated with the regulation of antithrombin.

Differential expression of specific miRNAs have been observed in patient with venous thromboembolism. Starikova et al., “Differential expression of plasma miRNAs in patients with unprovoked venous thromboembolism and healthy control individuals” Thrombosis Research 136:566-572 (2015). After screening over seven hundred miRNAs, the reference identifies only nine miRNAs that are overexpressed during venous thromboembolism and none of these are contemplated to modulate a SERPINC1 gene. Furthermore, the reference fails to identify miR-141-3p, miR-200a-3p, miR-200b-5p or miR-200c-3p.

B. Gene Expression

miRNAs are small (e.g., approximately 18-25 nucleotides), endogenous, non-coding RNAs that are involved in messenger RNA (mRNA) silencing. miRNAs have been reported to be involved in the post-transcriptional regulation of gene expression in a large number of physiologic and pathologic processes.¹²⁻¹⁴ For example, miRNAs have been demonstrated to regulate the expression of approximately 90% of human genes, and increasing evidence suggests that miRNAs may play a role in intracellular events including, but not limited to, cell differentiation, cell cycle regulation, metabolism and/or apoptosis.¹²⁻¹⁵ miRNAs have also been studied and documented in various human conditions including, but not limited to, cancers, cardiovascular diseases, autoimmune conditions, and psychiatric disorders.¹³ In one embodiment, the present invention contemplates a method comprising miRNA regulation of hemostasis. Although it is not necessary to understand the mechanism of an invention it is believed that miRNA expression modulates gene expression to regulate coagulation hemostasis.

miRNAs are believed to modulate gene expression by promoting silencing or degradation of mRNA that ultimately inhibits translation of the protein encoded by the miRNA-modulated gene.¹²⁻¹⁵ A single miRNA may target multiple genes or multiple miRNAs that may function cooperatively to regulate a single gene and its pathway.¹² While the majority of miRNAs exist intracellularly, a large number exist extracellularly and can be reliably detected in blood plasma and/or other body fluids.¹³ Variations in circulating levels of miRNAs may therefore provide valuable information about representative changes of overall intracellular miRNA signatures and their gene targets.

C. Thrombosis

Although more than two decades have passed since the first description of miRNAs, these small, non-coding RNAs have only recently gained attention in the study of hemostasis. They were first associated with platelet function and most of the existing literature has investigated their role in arterial thrombosis.²² More than 500 miRNAs have been defined in platelets and while some have been described in the regulation of fibrinogen, protein S, tissue factor and factor XI, their specific roles in hemostasis remains poorly understood.^{14,22}

In a recent review of the literature by Jiang et al. (2017), only three published articles were identified that examined miRNA expression in patients with VTE.²³ Overall, these three articles included a total of 87 patients with VTE (e.g., DVT or PE) and 219 controls and identified 13 differentially expressed miRNAs among them (eight upregulated and five downregulated in patients with VTE). Upregulated miRNAs included; miR-424-5p, miR-582, miR-195, miR-532, miR-10b-5p, miR-320a, miR-320b, and miR-423-5p. Downregulated miRNAs included: miR-136-5p, miR-103a-3p, miR-191-5p, miR-301a-3p, and miR-199b-3p.²³ MiR-424-5p was the only miRNA that was identified in more than one study.^{13,23,24} Although the sample populations for each study were vastly heterogeneous, there is a clear lack of reproducible findings in the existing literature with regard to miRNAs in VTE.^{13,23-25} Furthermore, these studies measured miRNA expression as biomarkers, but did not clearly associate miRNAs with mRNA targets or proteins relevant to hemostasis. While the use of miRNAs as biomarkers for VTE may be limited, their expression profiles may be more useful in deciphering the regulatory dynamics involved in hemostasis, ultimately balancing coagulation and thrombosis with bleeding.^{15,25,26}

The present data shows that miR-320b, which was upregulated in previous studies of patients with unprovoked VTE, was downregulated hibernating black bears. miR-320b appears to be highly expressed in human platelets and has been associated with mRNA targets involved in platelet function including, but not limited to, intracellular adhesion molecule 1 (ICAM1), pyruvate dehydrogenase kinase isozyme 3 (PDK3), phosphatidylinositol 3-kinase (PIK3R1) and integrin beta-3 precursor (ITGB3).²⁴ In addition to being a putative biomarker for VTE and regulator of platelet function, miR-320b appears to play a role in the maintenance of hemostasis in hibernating black bears.^{13,23,24,26,27}

MiR-320c has also been studied in platelet disorders has been found to be upregulated in patients with immune thrombocytopenic purpura (ITP). ITP is an autoimmune condition in which a reduced quantity of circulating platelets leads to severe bruising and/or bleeding.²⁷ Furthermore, in mammalian cell lines, miR-320c expression was associated with decreased platelet cell activation, through modulation of RAP1 (Ras-related protein 1).²⁸ Interestingly, the presently disclosed data show that miR-320c was downregulated during hibernation. In addition to being a putative biomarker for VTE and regulator of platelet function, miR-320c appears to play a role in the maintenance of hemostasis in the hibernating black bears.^{13,23,24,26,27}

Additionally, miR-320d was downregulated in hibernating versus active black bears. miR-320d has been studied as a biomarker in various human cancers and has also been found to play an important role in the migration and metastasis of tumor cells²⁹ through the regulation of proteins including, but not limited to, matrix metalloproteinase 2 (MMP2), matrix metalloproteinase 9 (MMP9), epithelial cadherin (E-cadherin), neural cadherin (N-cadherin) and integrin-β1 expression.³⁰ While miR-320d's role in platelet and endothelial cell function has not been defined, it is plausible that through the regulation of the aforementioned cell surface receptors and cell adhesion molecules, miR-320d may also play a role in hemostasis in hibernating black bears.

D. Hibernation

An miRNA expression profile was compared to known bear genome sequences. The data presented herein revealed that fifteen (15) miRNAs were differentially expressed in a statistically significant manner between hibernating and active American black bears. See, Table 1.

TABLE 1
Statistically Significant Differential
Expression Of miRNA In Black Bears

| | Direction of expression | | Fold Change | |
|---|---|---|---|---|
| miRNA | Hibernating (H) | Active (A) | (H:A) | P-adj |
| miR10b-3p | ↓ | ↑ | 0.283 | 0.005 |
| miR-136-3p | ↓ | ↑ | 0.136 | <0.001 |
| miR-181c-5p | ↓ | ↑ | 0.120 | <0.001 |
| miR-200a-3p | ↓ | ↑ | 0.203 | <0.001 |
| miR-200b-5p | ↓ | ↑ | 0.910 | <0.001 |
| miR-200c-3p | ↓ | ↑ | 0.136 | 0.002 |
| miR-320b | ↓ | ↑ | 0.133 | <0.001 |
| miR-320c | ↓ | ↑ | 0.112 | <0.001 |
| miR-320d | ↓ | ↑ | 0.240 | <0.001 |
| miR-15a-5p | ↑ | ↓ | 3.59 | <0.001 |
| miR-15b-3p | ↑ | ↓ | 4.87 | <0.001 |
| miR-15b-5p | ↑ | ↓ | 4.08 | <0.001 |
| miR-16-5p | ↑ | ↓ | 14.05 | <0.001 |
| miR-92a-3p | ↑ | ↓ | 3.27 | 0.003 |
| miR-150-5p | ↑ | ↓ | 4.13 | <0.001 |

Of the fifteen, nine (9) miRNAs (e.g., miR10b-3p, miR-136-3p, miR-181c-5p, miR-200a-3p, miR-200b-5p, miR-200c-3p, miR-320b, miR-320c, and miR-320d) were downregulated in the hibernating bears while conversely upregulated in summer active bears. On the other hand, six (6) miRNAs (e.g., miR-15a-5p, miR-15b-3p, miR-15b-5p, miR-16-5p, miR-92a-3p, and miR-150-5p) were upregulated in hibernating bears while conversely downregulated in active bears.

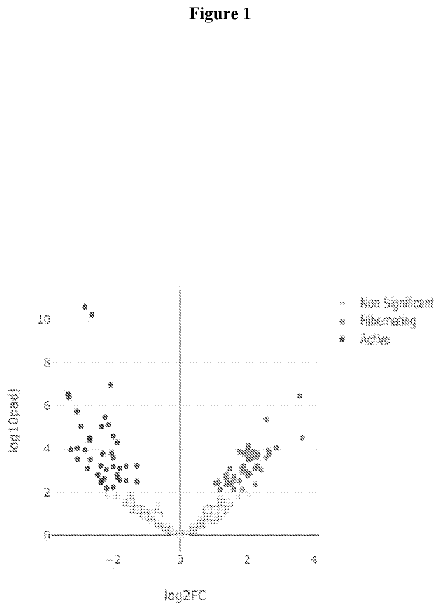

Given that the bear genome has not been completely cataloged, further quantification was performed including black bear miRNAs that displayed high similarity to human miRNA sequences. For example, by using nucleic acid sequences from known human miRNAs, an analysis confirmed differential expression of twelve (12) of the above fifteen (15) significantly differentially expressed miRNAs. This analysis also lead to an identification of an additional seventy-seven (77) differentially expressed miRNAs in the hibernating black bear as compared to active black bears, for a combined total of eighty-nine (89) observed differentially expressed miRNAs that reached levels of statistical significance. See, FIG. 1. Of these eighty-nine (89), forty (40) miRNAs were downregulated in hibernating bears while conversely upregulated in active bears. See, Table 2.

TABLE 2
Significantly Downregulated MiRNAs In Hibernating Black Bears

| | Direction of expression | | Fold Change | |
|---|---|---|---|---|
| miRNA | Hibernating (H) | Active A) | (H:A) | P-adj |
| miR-100-5p | ↓ | ↑ | 0.285415423 | 0.000799984 |
| miR-122-5p | ↓ | ↑ | 0.207396564 | 0.002310303 |
| miR-1224-5p | ↓ | ↑ | 0.128683193 | 9.10E−06 |
| miR-127-3p | ↓ | ↑ | 0.118679564 | 1.83E−06 |
| miR-136-3p | ↓ | ↑ | 0.148173851 | 0.000786485 |
| miR-141-3p | ↓ | ↑ | 0.200623271 | 0.000166479 |
| miR-143-5p | ↓ | ↑ | 0.104191282 | 0.000105832 |
| miR-152 | ↓ | ↑ | 0.250410587 | 0.000248927 |
| miR-181c-5p | ↓ | ↑ | 0.118664569 | 9.24E−05 |
| miR-193a-5p | ↓ | ↑ | 0.410246189 | 0.003287652 |
| miR-193b-5p | ↓ | ↑ | 0.242163125 | 0.000160306 |
| miR-194-5p | ↓ | ↑ | 0.287354811 | 0.002818766 |
| miR-199a-3p | ↓ | ↑ | 0.250971477 | 0.00065031 |
| miR-199a-5p | ↓ | ↑ | 0.154323908 | 3.03E−05 |
| miR-199b-3p | ↓ | ↑ | 0.274565735 | 0.002179728 |
| miR-200a-3p | ↓ | ↑ | 0.192663216 | 0.000589309 |
| miR-200b-5p | ↓ | ↑ | 0.198047369 | 0.002818766 |
| miR-200c-3p | ↓ | ↑ | 0.271946196 | 0.001598756 |
| miR-205-5p | ↓ | ↑ | 0.237032945 | 1.10E−07 |
| miR-21-5p | ↓ | ↑ | 0.409989292 | 0.000598064 |
| miR-214-3p | ↓ | ↑ | 0.139861413 | 0.000112052 |
| miR-215 | ↓ | ↑ | 0.10011394 | 4.11E−07 |
| miR-216a | ↓ | ↑ | 0.192928104 | 0.003592161 |
| miR-22-3p | ↓ | ↑ | 0.227247274 | 7.55E−06 |
| miR-299-3p | ↓ | ↑ | 0.24995862 | 0.00619532 |
| miR-320b | ↓ | ↑ | 0.161519879 | 6.42E−11 |
| miR-320c | ↓ | ↑ | 0.139371583 | 2.58E−11 |
| miR-320d | ↓ | ↑ | 0.272589175 | 5.08E−05 |
| miR-365a-5p | ↓ | ↑ | 0.219766755 | 0.006629359 |
| miR-375 | ↓ | ↑ | 0.15460477 | 3.77E−05 |
| miR-378a-3p | ↓ | ↑ | 0.326947112 | 0.000643366 |
| miR-409-3p | ↓ | ↑ | 0.211710994 | 3.40E−06 |
| miR-432-5p | ↓ | ↑ | 0.098237561 | 2.97E−07 |
| miR-574-3p | ↓ | ↑ | 0.250913792 | 2.62E−05 |
| miR-671-5p | ↓ | ↑ | 0.198149234 | 9.23E−06 |
| miR-885-3p | ↓ | ↑ | 0.217664416 | 0.000929788 |
| miR-9-5p | ↓ | ↑ | 0.118998883 | 0.000299894 |
| miR-95 | ↓ | ↑ | 0.156045229 | 0.000313997 |
| miR-99a-5p | ↓ | ↑ | 0.327206853 | 0.003004004 |
| miR-99b-3p | ↓ | ↑ | 0.182644403 | 0.001606912 |

On the other hand, forty-nine (49) miRNAs were significantly upregulated in hibernating bears while conversely downregulated in active bears. See, Table 3.

TABLE 3
Significantly Upregulated miRNAs In Hibernating Black Bears

| | Direction of expression | | Fold Change | |
|---|---|---|---|---|
| miRNA | Hibernating (H) | Active (A) | (IRA) | P-adj |
| let-7a-5p | ↑ | ↓ | 4.123008931 | 7.45E−05 |
| let-7b-5p | ↑ | ↓ | 2.615392943 | 0.00200948 |
| let-7d-5p | ↑ | ↓ | 3.614678796 | 0.001153379 |
| let-7f-5p | ↑ | ↓ | 4.33164442 | 0.000162019 |
| let-7g-5p | ↑ | ↓ | 4.011266861 | 0.000112052 |
| let-7i-5p | ↑ | ↓ | 2.605675874 | 0.00253792 |
| miR-101-3p | ↑ | ↓ | 2.073611471 | 0.004187173 |
| miR-103a-3p | ↑ | ↓ | 5.955881131 | 4.21E−06 |
| miR-106a-5p | ↑ | ↓ | 2.571491856 | 0.004547422 |
| miR-106b-3p | ↑ | ↓ | 3.031801284 | 0.007038822 |
| miR-106b-5p | ↑ | ↓ | 5.363525105 | 0.000936504 |
| miR-107 | ↑ | ↓ | 2.290148508 | 0.007355849 |
| miR-126-3p | ↑ | ↓ | 3.044626926 | 0.003057395 |
| miR-128 | ↑ | ↓ | 3.9483463 | 0.000162064 |
| miR-142-5p | ↑ | ↓ | 4.165321943 | 0.000248927 |
| miR-144-5p | ↑ | ↓ | 4.766211562 | 0.004504046 |
| miR-148b-3p | ↑ | ↓ | 2.187338184 | 0.004113578 |
| miR-150-5p | ↑ | ↓ | 3.737619361 | 0.000159417 |
| miR-15a-5p | ↑ | ↓ | 3.683817802 | 0.000598064 |
| miR-15b-3p | ↑ | ↓ | 4.910269899 | 0.000594873 |
| miR-15b-5p | ↑ | ↓ | 3.687071453 | 0.001242587 |
| miR-16-5p | ↑ | ↓ | 11.97199504 | 3.52E−07 |
| miR-17-5p | ↑ | ↓ | 4.978349131 | 0.000166479 |
| miR-182-5p | ↑ | ↓ | 12.57703387 | 3.03E−05 |
| miR-185-5p | ↑ | ↓ | 2.741165511 | 0.004504046 |
| miR-186-5p | ↑ | ↓ | 4.117878991 | 0.000313997 |
| miR-18a-5p | ↑ | ↓ | 3.415600273 | 0.003057395 |
| miR-191-5p | ↑ | ↓ | 7.330371328 | 8.75E−05 |
| miR-19a-3p | ↑ | ↓ | 4.169543676 | 0.00024524 |
| miR-20a-5p | ↑ | ↓ | 3.609875959 | 0.000725564 |
| miR-223-3p | ↑ | ↓ | 3.038191229 | 0.00200948 |
| miR-25-3p | ↑ | ↓ | 4.447413003 | 0.000242373 |
| miR-26a-5p | ↑ | ↓ | 2.61498009 | 0.001567728 |
| miR-26b-5p | ↑ | ↓ | 3.422102888 | 0.000132193 |
| miR-30c-5p | ↑ | ↓ | 6.271530464 | 0.000114238 |
| miR-3200-3p | ↑ | ↓ | 3.624179175 | 0.007540383 |
| miR-335-5p | ↑ | ↓ | 3.957697175 | 0.001062716 |
| miR-339-5p | ↑ | ↓ | 6.316483141 | 0.000166479 |
| miR-374a-5p | ↑ | ↓ | 2.853197392 | 0.004113578 |
| miR-421 | ↑ | ↓ | 4.22090786 | 0.001435446 |
| miR-425-3p | ↑ | ↓ | 4.793915503 | 0.000248927 |
| miR-425-5p | ↑ | ↓ | 4.26458144 | 0.000266505 |
| miR-451a | ↑ | ↓ | 4.7103625 | 0.000786485 |
| miR-454-3p | ↑ | ↓ | 5.952502497 | 0.000250837 |
| miR-484 | ↑ | ↓ | 4.013628852 | 0.001678732 |
| miR-744-5p | ↑ | ↓ | 2.230132357 | 0.003300553 |
| miR-92a-3p | ↑ | ↓ | 2.82097716 | 0.000823742 |
| miR-93-5p | ↑ | ↓ | 4.602482283 | 0.000132193 |
| miR-98 | ↑ | ↓ | 4.886726405 | 0.000248927 |

### IV. Antisense Therapy

Antisense technology has very recently proven to be useful in the prevention of VTE in post-surgical patients. A recently published trial of 300 patients undergoing elective total knee arthroplasty were randomized to receive either an antisense-oligonucleotide against factor XI (FXI-ASO) or enoxaparin for prevention of post-operative VTE.³⁸ Treatment with the anti-sense FXI-ASO at either 200 mg or 300 mg was associated with lower levels of factor XI as compared to enoxaparin. However, VTE rates were equal to enoxaparin at 200 mg FXI-ASO but were significantly lower at 300 mg FXI-ASO. Additionally, although there was no statistically significant difference in rates of post-operative bleeding, there was a trend toward decreased bleeding in the groups that received FXI-ASO.³⁸

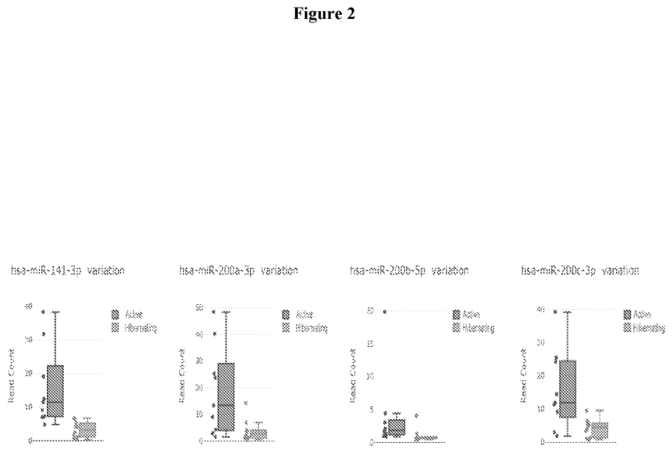

In order to identify gene targets for these seventy-seven (77) miRNAs, total RNA and mRNA was sequenced from black bear kidney cells collected before and soon after hibernation. (Jackson Laboratory, Bar Harbor, Me.).²⁰ These data showed that the SERPINC1 gene was upregulated soon after hibernation but downregulated during the active state and was designated to be a differentially expressed protein coding gene²⁰. The data presented herein shown that the SERPINC1 gene is a target site for at least four of the above identified differentially expressed miRNAs (e.g., miR-141-3p, miR-200a-3p, miR-200b-5p, and miR-200c-3p). See, FIG. 2. The data shown herein demonstrate that miR-141-3p, miR-200a-3p, miR-200b-5p, and miR-200c-3p were all downregulated during hibernation and upregulated during summer active state.

The Cytokine-Inducible SH2-Containing Protein (CISH) gene and the Solute Carrier Family 16, Member 1 (Monocarboxylic Acid Transporter 1; SLC16A1) gene were also identified as targets for differentially expressed miRNAs miR-22-3p, miR-128 and miR-216. For example, miR-22-3p was downregulated during hibernation and found to target the CISH gene. The CISH gene was observed to be upregulated soon after hibernation. On the other hand, miR-128 was upregulated during hibernation and targets LSC16A1. Interestingly, miR-216 also targets LSC15A1 but was observed to be downregulated during hibernation. The LSC16A1 gene was observed to be upregulated soon after hibernation. While many differentially expressed miRNAs were identified above between hibernating and active black bears, the present data identifies at least two additional gene targets besides SERPINC1. The CISH gene is regulated by miR-22-3p and encodes a cytokine suppressing protein²⁰. The SLC16A1 gene is regulated by both miR-128 and miR-216 and encodes a monocarboxylate transporter protein (MCT1).³⁹ Neither of these genes have been studied in hemostasis and their role in anticoagulation in the hibernating bear is currently unknown. These data suggest that carefully selected miRNA binding sites may serve as targets for the development of novel therapeutic agents designed to modulate the expression of genes involved in hemostasis, such as SERPINC1 to regulate the translation of anticoagulant proteins such as AT.

Several other miRNAs that were differentially expressed between hibernating and active black bears were not found herein to be associated with any SERPINC1-related mRNA targets. However, nearly all of these non-SERPINC1-related miRNAs have been extensively studied in the development and progression of various tumors and many have been recognized as biomarkers for various human cancers.^{29,40-50} They have most commonly been studied in cancers of the nasopharynx, lung, breast, stomach, pancreas, liver, colon, prostate, as well as in lymphomas, leukemias, gliomas and melanomas.^{29,30,40-45,47-58} They have been associated with numerous different mRNA targets and genetic pathways that regulate mitochondrial mediated apoptosis, tumor cell proliferation, migration and invasion, tumor metastasis and recurrence.⁵⁹

### V. Antisense Inhibitors

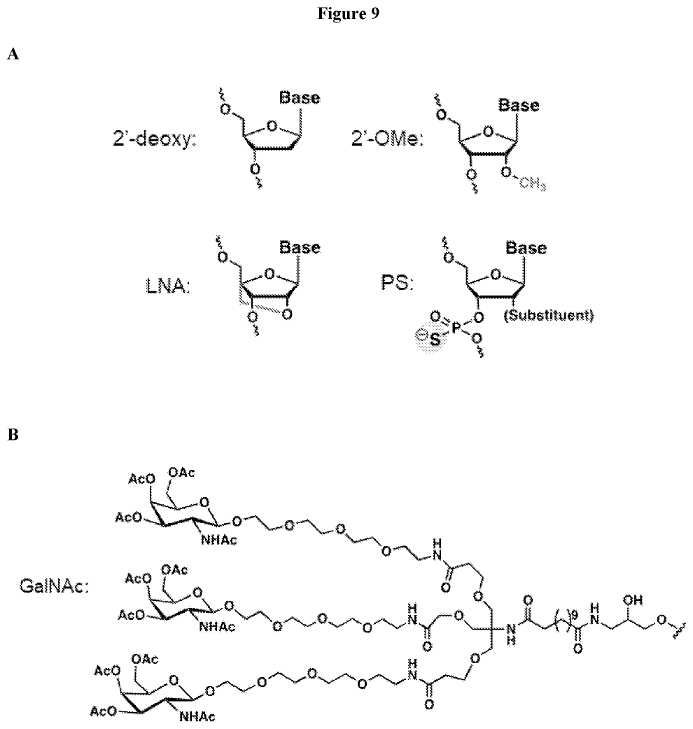

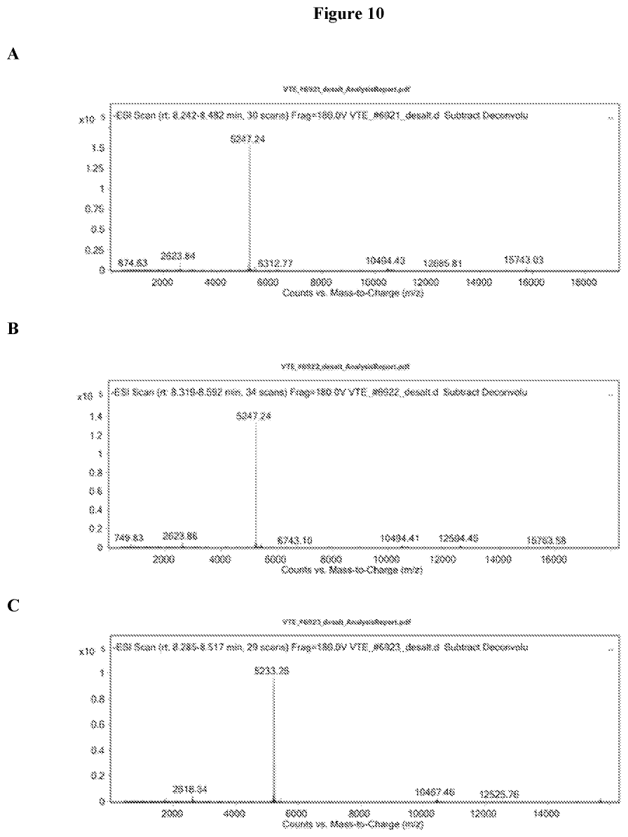

In one embodiment, the present invention contemplates a composition comprising an anti-miR oligonucleotide (e.g., an antisense inhibitor). In one embodiment, the antisense inhibitor comprises a plurality of nucleic acids conjugated by a phosphorothioate linker. In one embodiment, at least one of the plurality of nucleic acids comprises a modification. In one embodiment, the modification includes, but is not limited to, a 2′-OMe modification, a locked nucleic acid (LNA) modification or a 2′-deoxy modification. See, FIG. 9A. In one embodiment, the 3′ end of the antisense inhibitor comprises a GalNAc conjugate. See, FIG. 9B. In one embodiment, the antisense inhibitor includes, but is not limited to, anti200bc, anti200a+141, anti429, anti18a and/or anti19b.

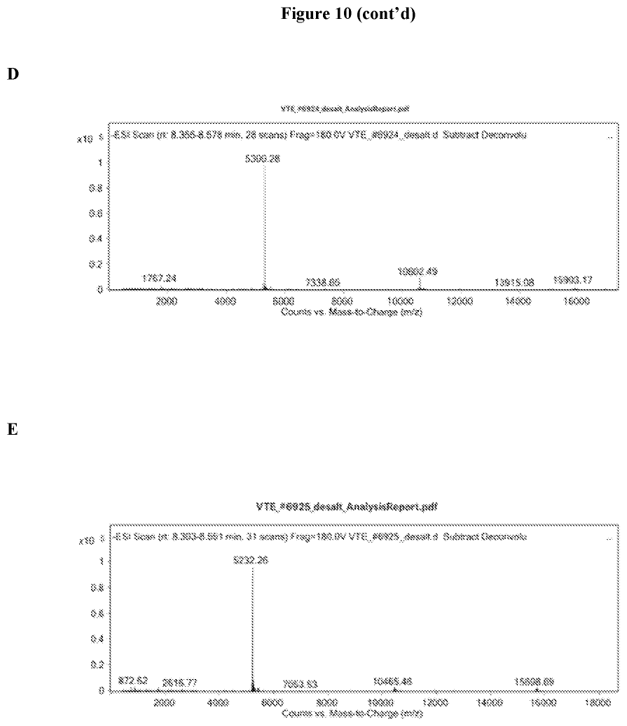

The structure of each antisense inhibitor was validated by LC-mass spectroscopy. See, FIG. 10A-E. A summary of these specific sequences and mass properties for these inhibitors are shown below. See, Table 4.

TABLE 4
Sequence of anti-miR oligonucleotides

| | | | Mass calcd | Mass | ^{a}ε(calcd) |
|---|---|---|---|---|---|
| Oligo# | Target | Sequence (5′ − >3′)* | [M − h]⁻ | found | L/mol · cm |
| 6921 | anti200bc | (mG)#(lG)#(mC)#(lA)#(lG)#(lT)#(lA)#(mU)#(mU)(dA)- | 5246.5 | 5247.24 | 107700 |
| | | GalNac | | | |
| 6922 | anti200a + | (mG)#(lA)#(mC)#(lA)#(lG)#(lT)#(lG)#(mU)#(mU)(dA)- | 5246.5 | 5247.24 | 107600 |
| | 141 | GalNac | | | |
| 6923 | anti429 | (mG)#(lA)#(mC)#(lA)#(lG)#(lT)#(lA)#(mU)#(mU)(dA)- | 5232.5 | 5233.25 | 111000 |
| | | GalNac | | | |
| 6924 | anti18a | (mU)#(lT)#(mA)#(lG)#(lG)#(lG)#(lC)#(mA)#(mG)(dA)- | 5299.6 | 5300.28 | 106300 |
| | | GalNac | | | |
| 6925 | anti19b | (mG)#(lA)#(mU)#(lT)#(lT)#(lG)#(lC)#(mA)#(mC)(dA)- | 5231.6 | 5232.26 | 100200 |
| | | GalNac | | | |

*(mN): 2′-OMe, (lN): LNA, (dN): 2′-deoxy,
#Phosphorothioate, 3′-end has GalNAc conjugate

Synthesis of these anti-mR oligonucleotides was performed with a MerMade 12 automated RNA synthesizer (BioAutomation) on a 1 μmol GalNAc (TEG)CPG support (PRIMETECH, ALC.). All 2′-OMe and locked nucleic acid (LNA) phosphoramidites (ChemGenes, Co.) were prepared at a concentration of 0.1 M in anhydrous CH3CN except for 2′-OMe-Ur and LNA-C where CH3CN-DMF (85:15, v/v) and CH3CN-DMF (1:1, v/v) were used, respectively. Synthesis was conducted on a standard 1.0 μmol scale RNA phosphoramidite synthesis cycle, which consists of (i) detritylation, (ii) coupling, (iii) capping, and (iv) iodine oxidation to phosphate by 0.02 M I2 in THF-pyridine-H₂O (7:2:1, v/v/v), or sulfurization by 0.1 M DDTT in pyridine:CH3CN (9:1, v/v). Coupling of phosphoramidites was conducted with a standard protocol for 2-cyanoethyl phosphoramidite using 5-(Benzylthio)-1H-tetrazole (BTT) as an activator. Each oligonucleotide was treated with 40% Methylamine-28% NH4OH (1:1, v/v) for 2 h at ambient temperature for deprotection and cleavage of oligonucleotides from solid support. Crude oligonucleotides were purified by standard anion exchange HPLC. All of obtained purified oligonucleotides were desalted by Sephadex G-25 (GE Healthcare) and characterized by electrospray ionization mass spectrometry (ESI-MS) analysis.

### VI. Messenger RNA Therapy

In one embodiment, the present invention contemplates a method providing a patient exhibiting at least one symptom of a thrombotic disorder and comprising a mutated SERPINC1 gene. In one embodiment, the SERPINC1 gene expresses an abnormal antithrombin protein. In one embodiment, the method further comprises administration of an SERPINC1 messenger ribonucleic acid sequence (mRNA). In one embodiment, the method further comprises translating said SERPINC1 mRNA sequence into an antithrombin protein. In one embodiment, the translated antithrombin results in the reduction of said at least one symptom of the thrombotic disorder. In one embodiment, a translated antithrombin from an exogenously delivered SERPINC1 mRNA sequence would essentially provide an intermediate term therapy option of activated SERPINC1 gene activity in the patient.

The administration of mRNA nucleic acids may be accomplished by the delivery of free mRNA into the patient, either locally or parenterally. Alternatively, the administration of mRNA nucleic acids may be delivered to the patient using a pharmaceutically acceptable carrier.

The present invention contemplates several pharmaceutically acceptable carriers that provide for roughly uniform distribution and have controllable rates of release. A variety of different media are described below that are useful in creating drug delivery systems. It is not intended that any one medium or carrier is limiting to the present invention. Note that any medium or carrier may be combined with another medium or carrier; for example, in one embodiment a polymer microparticle carrier attached to a compound may be combined with a gel medium.

Carriers or mediums contemplated by this invention comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

One embodiment of the present invention contemplates pharmaceutically acceptable carrier comprising mRNAs as described herein.

Microparticles

One embodiment of the present invention contemplates a medium comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids.

Liposomes

One embodiment of the present invention contemplates liposomes capable of attaching and releasing mRNAs as described herein. Liposomes are microscopic spherical lipid bilayers surrounding an aqueous core that are made from amphiphilic molecules such as phospholipids. For example, a liposome may trap an mRNA between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phosopholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One embodiment of the present invention contemplates an ultra high-shear technology to refine liposome production, resulting in stable, unilamellar (single layer) liposomes having specifically designed structural characteristics. These unique properties of liposomes, allow the simultaneous storage of normally immiscible compounds and the capability of their controlled release.

In some embodiments, the present invention contemplates cationic and anionic liposomes, as well as liposomes having neutral lipids. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

One embodiment of the present invention contemplates a medium comprising liposomes that provide controlled release of at least one mRNA. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes are broadly categorized into two classifications. Conventional liposomes are generally mixtures of stabilized natural lecithin (PC) that may comprise synthetic identical-chain phospholipids that may or may not contain glycolipids. Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

Microspheres, Microparticles And Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (Prolease®, Alkerme's: Cambridge, Mass.). For example, a freeze dried medium comprising at least one therapeutic agent is homogenized in a suitable solvent and sprayed to manufacture microspheres in the range of 20 to 90 μm. Techniques are then followed that maintain sustained release integrity during phases of purification, encapsulation and storage. Scott et al., Improving Protein Therapeutics With Sustained Release Formulations, Nature Biotechnology, Volume 16:153-157 (1998).

Modification of the microsphere composition by the use of biodegradable polymers can provide an ability to control the rate of mRNA release. Miller et al., Degradation Rates of Oral Resorbable Implants {Polylactates and Polyglycolates: Rate Modification and Changes in PLA/PGA Copolymer Ratios, J. Biomed. Mater. Res., Vol. II:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of a therapeutic agent is added to the biodegradable polymer metal salt solution. The weight ratio of an mRNA to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and mRNA is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and therapeutic agent mixture are: i) phase separation during a gradual addition of a coacervating agent; ii) an in-water drying method or phase separation method, where an antiflocculant is added to prevent particle agglomeration and iii) by a spray-drying method.

In one embodiment, the present invention contemplates a medium comprising a microsphere or microcapsule capable of delivering a controlled release of a therapeutic agent for a duration of approximately between 1 day and 6 months. In one embodiment, the microsphere or microparticle may be colored to allow the medical practitioner the ability to see the medium clearly as it is dispensed. In another embodiment, the microsphere or microcapsule may be clear. In another embodiment, the microsphere or microparticle is impregnated with a radio-opaque fluoroscopic dye.

Controlled release microcapsules may be produced by using known encapsulation techniques such as centrifugal extrusion, pan coating and air suspension. Such microspheres and/or microcapsules can be engineered to achieve desired release rates. For example, Oliosphere® (Macromed) is a controlled release microsphere system. These particular microsphere's are available in uniform sizes ranging between 5-500 um and composed of biocompatible and biodegradable polymers. Specific polymer compositions of a microsphere can control the therapeutic agent release rate such that custom-designed microspheres are possible, including effective management of the burst effect. ProMaxx® (Epic Therapeutics, Inc.) is a protein-matrix delivery system. The system is aqueous in nature and is adaptable to standard pharmaceutical delivery models. In particular, ProMaxx® are bioerodible protein microspheres that deliver both small and macromolecular drugs, and may be customized regarding both microsphere size and desired release characteristics.

In one embodiment, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated compound, therefore, will be selected for the pH environment in which dissolution is desired and stored in a pH preselected to maintain stability. Examples of pH sensitive material useful as encapsulants are Eudragit® L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

In one embodiment, the present invention contemplates a microparticle comprising a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, an mRNA may be directly bound to the surface of the microparticle or indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

One having skill in the art will understand that the shape of the microspheres need not be exactly spherical; only as very small particles capable of being sprayed or spread into or onto a surgical site (i.e., either open or closed). In one embodiment, microparticles are comprised of a biocompatible and/or biodegradable material selected from the group consisting of polylactide, polyglycolide and copolymers of lactide/glycolide (PLGA), hyaluronic acid, modified polysaccharides and any other well known material.

### VII. Transfection of Therapeutic Nucleic Acids

In one embodiment, the present invention contemplates a method comprising, administering a nucleic acid to a patient having, or at risk for, a thrombotic event. In one embodiment, the nucleic acid is an antisense inhibitor. In one embodiment, the nucleic acid is a messenger RNA.

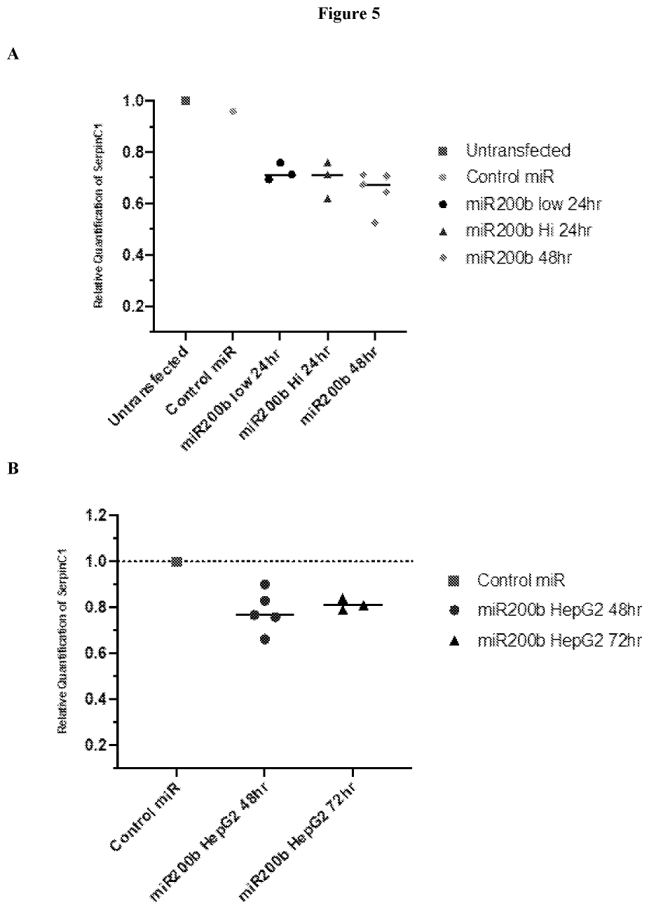

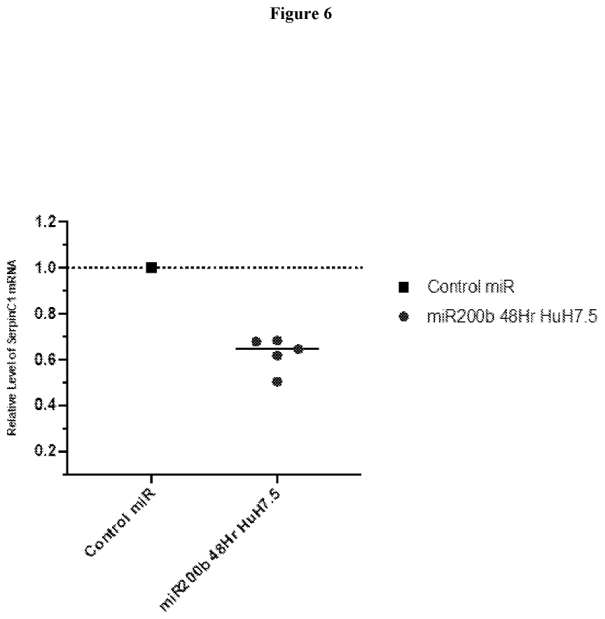

The data presented herein demonstrates that a composition of a nucleic acid (e.g., miR200b) with a transfection reagent comprising lipofectamine effectively reduces SERPINC1 expression in HuH7.5 cells. For example, two concentrations of the Lipofectamine 3000 reagent were compared with 1 μg of DNA on HuH7.5 cells at two different time points; 24 hrs and 48 hrs. The data shows that there is around 30% gene silencing seen for all conditions. See, FIG. 5A. To confirm this data, the miR200b nucleic acid was testing in the immortalized liver cell line, HepG2, but using the highest concentration of Lipofectamine tested above, 1 μg of DNA, and 48 hr and 72 hr incubations. The data shows there was around a 20% silencing of SERPINC1 expression. See FIG. 5B. The HuH7.5 cell transfection was repeated with the higher concentration of Lipofectamine for 48 hours. The data shows that when normalized to a control miRNA that does not target SerpinC1, between 30-40% silencing of SERPINC1 mRNA expression was observed. See, FIG. 6.

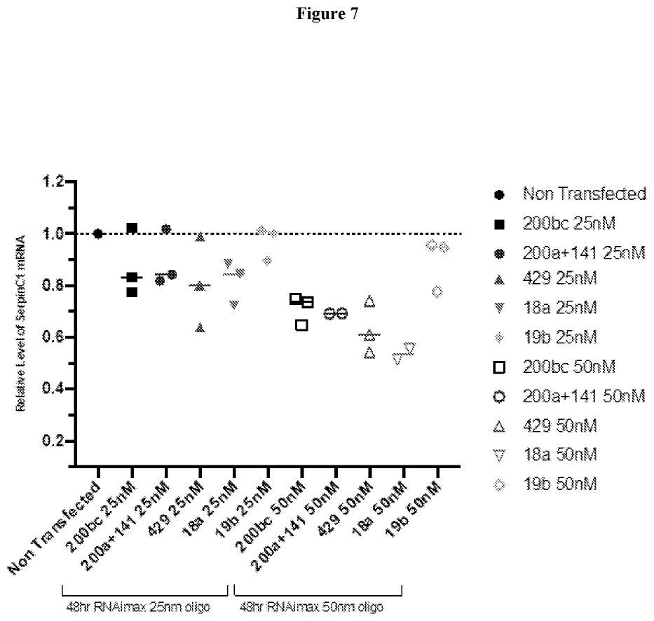

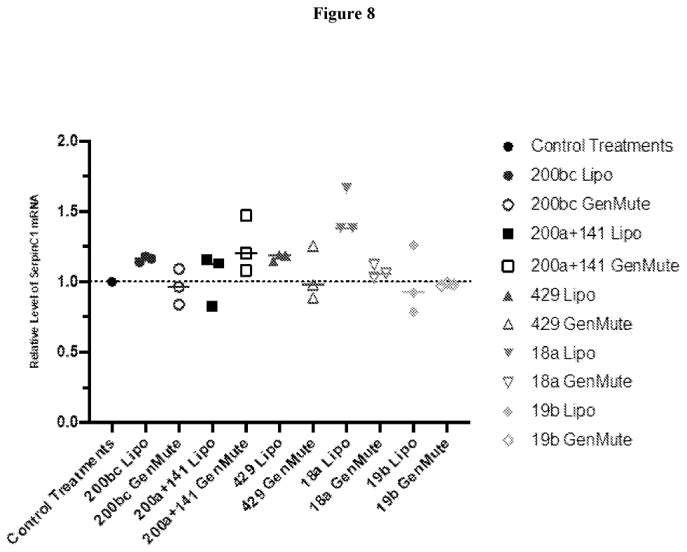

Subsequent to the above validation of this transfection method, five (5) antisense inhibitors were tested in the presence of the RNAimax transfection reagent for 48 hours in HuH.7.5 cells. The downregulation of SERPINC1 mRNA expression in these data suggest that RNAimax is toxic to these cells thereby preventing the expected response to the antisense inhibitors that upregulate mRNA expression. See, FIG. 7. Consequently, an alternative transfection reagent comprising GenMute or Lipofectamine was observed to result in the expected upregulation of SERPINC1 mRNA expression when the five (5) disclosed antisense inhibitors were incubated for 48 hours in Huh7.5 cells. It was determined that the antisense inhibitors amiR200bc, amiR429, and amiR 18a appeared to have the greatest efficacy. See, FIG. 8.

### VIII. Small Molecule Inhibitor Therapy

In one embodiment, the present invention contemplates a method providing a patient exhibiting at least one symptom of a thrombotic disorder and comprising a SERPINC1 gene, said SERPINC1 gene comprising at least one miRNA binding site. In one embodiment, the method further comprises a small molecule inhibitor that attaches to said at least one miRNA binding site. In one embodiment, the method further comprises increasing expression of antithrombin from said SERPINC1 gene. In one embodiment, the increased expression of antithrombin results in the reduction of said at least one symptom of the thrombotic disorder. In one embodiment, a small molecule inhibitor that can increase SERPINC1 gene expression would essentially provide a short term therapy option of activated SERPINC1 gene activity in the patient.

In some embodiments, small organic molecule drugs are identified using drug screening methods. In some embodiments, the present invention provides drug screening assays (e.g., to screen for drugs that bind to SERPINC1 miRNA binding sites). These small organic molecules may be discovered using any one of several high-throughput screening methods. Stockwell, B. R., “Exploring biology with small organic molecules” Nature 432:846-854 (2004); Kay et al., “High-throughput screening strategies to identify inhibitors of protein-protein interactions” Mol. Diversity 1:139-140 (1996); Pfleger et al., “Extended bioluminescence resonance energy transfer (eBRET) for monitoring prolonged protein-protein interactions in live cells” Cell Signaling 18:1664-1670 (2006); Jung et al., “Surface plasmon resonance imaging-based protein arrays for high-throughput screening of protein-protein interaction inhibitors” Proteomics 5: 4427-4431 (2005); Nieuwenhuijsen et al., “A dual luciferase multiplexed high-throughput screening platform for protein-protein interactions” J. Biomol. Screen 8:676-684 (2003); and Berg, T., “Modulation of protein-protein interactions with small organic molecules” Angew. Chem. Int. Ed. Engl. 42:2462-2481 (2003).

In some embodiments, the present invention provides drug screening assays (e.g., to screen for miRNA binding site inhibitor drugs). The screening methods of the present invention utilize gene expression maps identified using the methods of the present invention (e.g., including but not limited to, SERPINC1). For example, in some embodiments, the present invention provides methods of screening for compounds that alter (e.g., increase or decrease) the expression of SERPINC1 expression maps. In some embodiments, candidate compounds are antibodies that specifically bind to a protein encoded by a SERPINC1 gene of the present invention (e.g., for example, antithrombin).

In one screening method, candidate compounds are evaluated for their ability to alter SERPINC1 gene expression by contacting a compound with a cell expressing a thrombosis induced protein and then assaying for the effect of the candidate compounds on expression. In some embodiments, the effect of candidate compounds on expression of SERPINC1 gene is assayed for by detecting the level of mRNA expressed by the cell. mRNA expression can be detected by any suitable method. In other embodiments, the effect of candidate compounds on expression of SERPINC1 genes is assayed by measuring the level of antithrombin protein. The level of antithrombin expressed can be measured using any suitable method, including but not limited to, those disclosed herein.

Specifically, the present invention provides screening methods for identifying modulators, i.e., candidate or test compounds or agents (e.g., proteins, peptides, peptidomimetics, peptoids, small molecules or other drugs) which bind to antithrombin protein, have an inhibitory (or stimulatory) effect on, for example, SERPINC1 gene expression or gene product activity, or have a stimulatory or inhibitory effect on, for example, the expression or activity of antithrombin. Compounds thus identified can be used to modulate the activity of SERPINC1 gene products (e.g., antithrombin) either directly or indirectly in a therapeutic protocol, to elaborate the biological function of the antithrombin, or to identify compounds that disrupt normal SERPINC1 gene interactions. Compounds which inhibit the activity or expression of SERPINC1 genes are useful in the treatment of thrombosis disorders.

In one embodiment, the invention provides assays for screening candidate or test compounds that are substrates of an antithrombin protein or a biologically active portion thereof. In another embodiment, the invention provides assays for screening candidate or test compounds that bind to or modulate the activity of an antithrombin protein or a biologically active portion thereof.

The test compounds of the present invention can be obtained using any of the numerous approaches in combinatorial library methods, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678 85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Antivirus induced Drug Des. 12:145).

Numerous examples of methods for the synthesis of molecular libraries have been reported, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412 421 [1992]), or on beads (Lam, Nature 354:82 84 [1991]), chips (Fodor, Nature 364:555 556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386 390 [1990]; Devlin Science 249:404 406 [1990]; Cwirla et al., Proc. NatI. Acad. Sci. 87:6378 6382 [1990]; Felici, J. Mol. Biol. 222:301 [1991]).

In one embodiment, an assay is a cell-based assay in which a cell that expresses an antithrombin or biologically active portion thereof is contacted with a test compound, and the ability of the test compound to the modulate antithrombin protein activity is determined. Determining the ability of the test compound to modulate antithrombin protein activity can be accomplished by monitoring, for example, changes in enzymatic activity. The cell, for example, can be of mammalian origin.

The ability of the test compound to modulate an antithrombin protein binding to a compound, e.g., an antithrombin substrate, can also be evaluated. This can be accomplished, for example, by coupling the compound, e.g., a clotting factor protein, with a radioisotope or enzymatic label such that binding of the compound can be determined by detecting the labeled compound in a complex.

Alternatively, the antithrombin protein is coupled with a radioisotope or enzymatic label to monitor the ability of a test compound to modulate antithrombin protein binding to a substrate in a complex. For example, antithrombin protein can be labeled with 125I, 35S, 14C, or 3H, either directly or indirectly, and the radioisotope detected by direct counting of radioemission or by scintillation counting. Alternatively, antithrombin can be enzymatically labeled with, for example, horseradish peroxidase, alkaline phosphatase, or luciferase, and the enzymatic label detected by determination of conversion of an appropriate substrate to product.

The ability of a test compound to interact with an antithrombin protein with or without the labeling of any of the interactants can be evaluated. For example, a microphysiometer can be used to detect the interaction of a test compound with an antithrombin marker without the labeling of either the test compound or the antithrombin (McConnell et al. Science 257:1906 1912 [1992]). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a test compound and antithrombin.

In yet another embodiment, a cell-free assay is provided in which an antithrombin protein or biologically active portion thereof is contacted with a test compound and the ability of the test compound to bind to the antithrombin protein or biologically active portion thereof is evaluated. Preferred biologically active portions of the antithrombin proteins to be used in assays of the present invention include fragments that participate in interactions with substrates or other proteins, e.g., fragments with high surface probability scores.

Cell-free assays involve preparing a reaction mixture of the SERPINC1 gene protein and the test compound under conditions and for a time sufficient to allow the two components to interact and bind, thus forming a complex that can be removed and/or detected. The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FRET binding event can be conveniently measured through standard fluorometric detection means well known in the art (e.g., using a fluorimeter).

In another embodiment, determining the ability of the antithrombin protein to bind to a clotting factor protein can be accomplished using real-time Biomolecular Interaction Analysis (BIA) (see, e.g., Sjolander and Urbaniczky, Anal. Chem. 63:2338 2345 [1991] and Szabo et al. Curr. Opin. Struct. Biol. 5:699 705 [1995]). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the antithrombin protein or the test substance is anchored onto a solid phase. The antithrombin/test compound complexes anchored on the solid phase can be detected at the end of the reaction. Preferably, the antithrombin protein can be anchored onto a solid surface, and the test compound, (which is not anchored), can be labeled, either directly or indirectly, with detectable labels discussed herein.

In order to conduct the assay, test compound is added to the coated surface containing the anchored antithrombin. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized test compound is pre-labeled, the detection of label immobilized on the surface indicates that complexes were formed. Where the previously non-immobilized test compound is not pre-labeled, an indirect label can be used to detect complexes anchored on the surface; e.g., using a labeled antibody specific for the antithrombin protein (the antibody, in turn, can be directly labeled or indirectly labeled with, e.g., a labeled anti-IgG antibody).

This assay may be performed utilizing antibodies reactive with an antithrombin protein or clotting factor target but which do not interfere with binding of the antithrombin protein to its clotting factor target. Such antibodies can be derivatized to the wells of the plate, and unbound target or virus induced markers protein trapped in the wells by antibody conjugation. Methods for detecting such complexes, in addition to those described above for the GST-immobilized complexes, include immunodetection of complexes using antibodies reactive with the antithrombin protein or clotting factor target, as well as enzyme-linked assays which rely on detecting an enzymatic activity associated with the antithrombin protein or the clotting factor target.

Alternatively, cell free assays can be conducted in a liquid phase. In such an assay, the reaction products are separated from unreacted components, by any of a number of standard techniques, including, but not limited to: differential centrifugation (see, for example, Rivas and Minton, Trends Biochem Sci 18:284 7 [1993]); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology 1999, J. Wiley: New York). Such resins and chromatographic techniques are known to one skilled in the art (See e.g., Heegaard J. Mol. Recognit 11: 141 8 [1998]; Hageand Tweed J. Chromatogr. Biomed. Sci. Appl 699:499 525 [1997]). Further, fluorescence energy transfer may also be conveniently utilized, as described herein, to detect binding without further purification of the complex from solution.

The screening assay can include contacting the antithrombin protein or biologically active portion thereof with a known compound that binds the antithrombin protein to form an assay mixture, contacting the assay mixture with a test compound, and determining the ability of the test compound to preferentially bind to antithrombins or biologically active portion thereof, or to modulate the activity of a clotting factor protein, as compared to the known compound. To the extent that antithrombins can, in vivo, interact with one or more cellular or extracellular macromolecules, such as proteins, inhibitors of such an interaction are useful. A homogeneous assay can be used can be used to identify inhibitors.

For example, a preformed complex of the antithrombin protein and the interactive cellular or extracellular clotting factor protein is prepared such that either the antithrombins or the clotting factor protein targets are labeled, but the signal generated by the label is quenched due to complex formation (see, e.g., U.S. Pat. No. 4,109,496, herein incorporated by reference, that utilizes this approach for immunoassays). The addition of a test substance that competes with and displaces one of the species from the preformed complex will result in the generation of a signal above background. In this way, test substances that disrupt clotting factor protein/antithrombin protein binding partner interactions can be identified. Alternatively, antithrombin protein can be used as a “bait protein” in a two-hybrid assay or three-hybrid assay (see, e.g., U.S. Pat. No. 5,283,317; Zervos et al., Cell 72:223 232 [1993]; Madura et al., J. Biol. Chem. 268.12046 12054 [1993]; Bartel et al., Biotechniques 14:920 924 [1993]; Iwabuchi et al., Oncogene 8:1693 1696 [1993]; and Brent WO 94/10300; each of which is herein incorporated by reference), to identify other proteins, that bind to or interact with antithrombins.

Modulators of SERPINC1 gene expression can also be identified. For example, a cell or cell free mixture is contacted with a candidate compound and the expression of a SERPINC1 mRNA or antithrombin protein may be evaluated relative to the level of expression in the absence of the candidate compound. When SERPINC1 expression is greater (i.e., statistically significantly greater) in the presence of the candidate compound than in its absence, the candidate compound is identified as a stimulator. Alternatively, when expression is less (i.e., statistically significantly less) in the presence of the candidate compound than in its absence, the candidate compound is identified as an inhibitor. The level of SERPINC1 mRNA or antithrombin protein expression can be determined by many methods that are well known in the art.

A modulating agent can be identified using a cell-based or a cell free assay, and the ability of the agent to modulate the activity of an antithrombin protein can be confirmed in vivo, e.g., in an animal such as an animal model for a cardiovascular thrombotic disease.

This invention further pertains to novel agents identified by the above-described screening assays. Accordingly, it is within the scope of this invention to further use an agent identified as described herein (e.g., an antithrombin modulating agent, an antithrombin specific antibody, or a clotting factor protein binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

### EXPERIMENTAL

The presently disclosed data was collected in collaboration with the Michigan Department of Natural Resources (Marquette, Mich.), the Animal Health Diagnostic Center at Cornell University (Ithaca, N.Y.) and the Jackson Laboratories (Bar Harbor, Me.). All experimental procedures described were approved by, and performed in accordance with relevant guidelines and regulations from, the University of Massachusetts Institutional Animal Care and Use Committee (IACUC) and Mississippi State University's IACUC (protocol 12-012). Two separate, non-paired, groups of adult bears (n=21) were studied. One in the summer active state from June and July 2017 (n=11) and one in the hibernating state during December and February 2018 (n=10).

### Example 1

### Plasma Collection

Blood draws were performed under anesthesia (intramuscular injection of tiletamine hydrochloride [HCL] and zolazepam HCL mixture; 7 mg/kg body mass; Telazol®, Fort Dodge Animal Health, Fort Dodge, Iowa, USA) via the femoral or jugular veins in yellow top BD Vacutainer ACD blood collection tubes (BD Vacutainer, cat #364606). 1.5 mL of blood was transferred and spun down 500 xg; 10 min; room temperature. The top layer was transferred and spun down at 2000 xg; 10 min; room temperature. Plasma top liquid layer (˜720 μl) was transferred and immediately frozen on dry ice until processing. Of note, during hibernation blood was kept at 25° C. immediately after draw in a mini-transportable incubator (United Lab Plastics, cat #ZL7-TP).

### Example 2

### Data Preparation

Plasma samples were stored frozen for batch analysis and thawed to 37° C. just before assay. The samples were processed at University of Massachusetts Medical School Biocore using standardized, highly-parallel sequencing pipelines. The reads were first evaluated for their overall quality using FastQC (bioinformatics.babraham.ac.uk/projects/fastqc/) and ION Torrent read qualities converted to Illumina phred scores. The reads were then filtered for average base quality scores below 15. UMIs were removed from sequences and appended to the read name to group and remove PCR duplicates. 3′ adapter is left on the sequence to remove later in the pipeline. The PCR duplicates were then removed using UMITools 0.5.4.¹⁶

### Example 3

### miRNA Quantification

Since there are no completed miRNA sequences for Ursus americanus, we used two strategies to quantify miRNAs.

First, mirDeep2 v2.0.0.8 was used to discover known and novel miRNAs from sequencing data.¹⁷ Then, 3′ adapter sequence was removed from all samples to process the data with mirDeep2. The reads were then aligned with mapper,pl using -e-h-j-l18-m-p-v-n parameters to reference bear genome after removing the reads less than 18 nt (Jackson Laboratories; hgwdev.recse.ucsc.edu/˜ifiddes/maine_blackbear/). MiRDeep2 was then used to quantify known and novel mature miRNAs. All known mature miRNAs were downloaded from miRbase (doi.org/10.1093/nar/gkt1181). The quantification results were consolidated in a table to be used in differential miRNA analysis of bears during hibernation versus in active state.

Second, a quantification of miRNAs was performed that displayed a high similarity to human miRNAs in order to perform mRNA target prediction. To quantify known human miRNAs Kraken v13-274 was used.¹⁸ First Reaper in Kraken was used to strip low quality bases and low complexity sequences and trim 3′ adapter. The sequences were then aligned using bowtie with—time-v 2—best-k 21—strata-m 20 parameters to all known small RNA sequences. All small RNA features were quantified using annotation set v12-164 in Kraken. A consolidated table was generated for miRNA quantifications for differential miRNA analysis.

### Example 4

### Differential miRNA Analysis

The tables generated from the two strategies described in Example 3 discovered differentially expressed miRNAs using DEBrowser v1.9.16 (doi.org/10.1101/399931). For differential gene expression DESeq2 package in DEBrowser was used.¹⁹

The goal of this differential miRNA expression analysis was to detect miRNAs whose difference in expression, when accounting for the variance within condition, is higher than expected by chance. Since only a subset of all miRNA can be detected in exosomes, expression is therefore a bimodal distribution; where absent miRNAs may have counts that result from experimental noise. These non-expressed miRNAs were then filtered out before performing differential expression analysis.

DESeq2 computes the probability of whether a miRNA is differentially expressed or not. DESeq2 calculates both a nominal and a multiple hypothesis corrected p-value (padj). To find significant differentially expressed miRNAs, the miRNAs having lower padj values and higher fold changes were selected for downstream analysis. Here, 0.01<for padj value and >2 fold change were used as cutoffs to detect differentially expressed miRNAs.

### Example 5

### Comparison of miRNA with mRNA Targets from Bear Kidneys

To find possible mRNA targets for the overexpressed miRNAs, total RNA that was sequenced from kidney cells of 3 female and 3 male black bears before hibernation (September 2015) and soon after hibernation (May and June 2015; The Korstanje Lab, The Jackson Lab, Bar Harbor Me.).²⁰

Additional methods regarding kidney sampling, library preparation total RNA sequencing are described elsewhere.²⁰ After data preparation steps, 3′ adapter of the paired end reads were removed using trimmomatic version 0.32. rRNA, miRNA, tRNA, and snRNA sequences were filtered out using Bowtie2, version 2.2.3 using reference sequences from bear. After these filtration and adapter removal steps, the reads were aligned to bear transcriptome and transcript level abundance and were computed using RSEM v1.3.0.²¹ DE analysis was then performed as defined in Example 4 for mRNAs. Since genes are not annotated in the bear genome, after we found DE genes, we used blast to find the genes deposited to NCBI (doi.org/10.1089/10665270050081478).

### Example 6

### miRNA Expression Construct

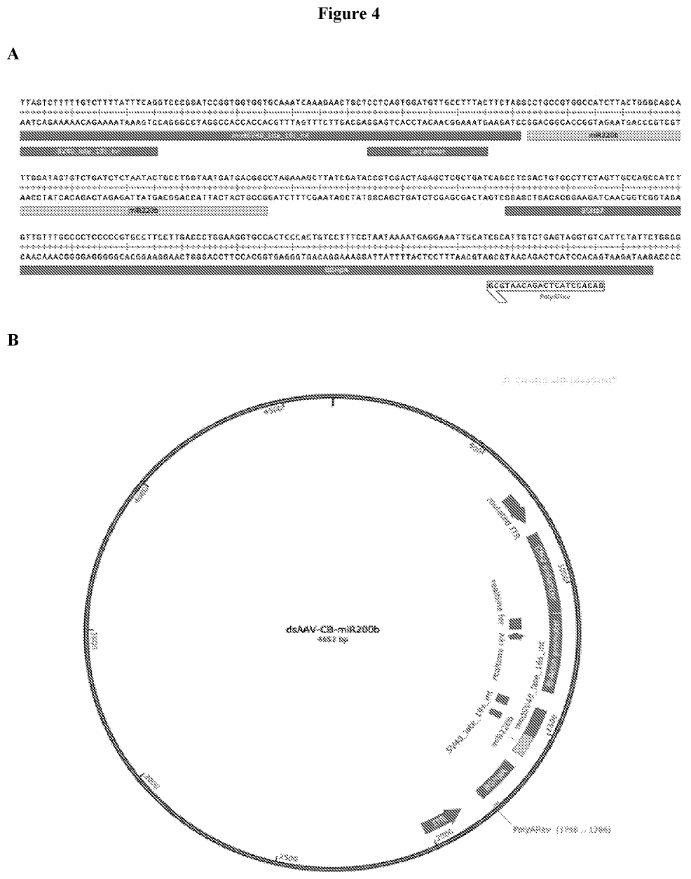

This example shows an exemplary construct design for a double stranded plasmid and a polII promoter to drive expression of an miR220b antisense molecule. The miR220b coding region was flanked by SV40_late_16s_int and BGHpA sequences. See, FIG. 4A. The plasmid was then packaged into an AAV8 vector. See, FIG. 4B.

### Example 7

### In Vivo Transfection with an Antisense Inhibitor

Nine (9) Mice were injected intravenously with an AAV8-miR200b vector and were divided equally into the following dose groups:
Group 1: 1e12vg total 4e13v g/kg
Group 2: 1e11vg total 4e12v g/kg
Group 3: 1e10vg total 4e11v g/kg
   A 0 week, 1 week, 2 week, 4 week, 6 week and 8 week blood determination of SERPINC1 mRNA expression will be determined. It is expected that the SERPINC1 mRNA expression will be downregulated.

A second experiment of similar design will intravenously inject an AAV8-antisense inhibitor. It is expected that SERPINC1 mRNA expression will be upregulated.

### REFERENCES

1 Friedrich, A. U. et al. Comparative coagulation studies in hibernating and summer-active black bears (Ursus americanus). Thromb. Res. 158, 16-18, doi:10.1016/j.thromres.2017.07.034 (2017).
2 Caprini, J. A. Risk assessment as a guide for the prevention of the many faces of venous thromboembolism. Am. J. Surg. 199, S3-10, doi:10.1016/j.amjsurg.2009.10.006 (2010).
3 Grosse, S. D., Nelson, R. E., Nyarko, K. A., Richardson, L. C. & Raskob, G. E. The economic burden of incident venous thromboembolism in the United States: A review of estimated attributable healthcare costs. Thromb. Res. 137, 3-10, doi:10.1016/j.thromres.2015.11.033 (2016).
4 Cohoon, K., Leibson, C., Ransom, J., Ashrani, A., Petterson, T., Long, K., Bailey, K., and Heit, J. Costs of Venous Thromboembolism Associated with Hospitalization for Medical Illness. Am. J. Manag. Care 21, 255-263 (2015).
5 Frobert, O. et al. Platelet function in brown bear (Ursus arctos) compared to man. Thromb J8, 11, doi:10.1186/1477-9560-8-11 (2010).
6 Martin, S. L. & Yoder, A. D. Theme and variations: heterothermy in mammals. Integr. Comp. Biol. 54, 439-442, doi:10.1093/icb/icu085 (2014).
7 Fedorov, V. B. et al. Elevated expression of protein biosynthesis genes in liver and muscle of hibernating black bears (Ursus americanus). Physiol. Genomics 37, 108-118, doi:10.1152/physiolgenomics.90398.2008 (2009).
8 Fedorov, V. B. et al. Modulation of gene expression in heart and liver of hibernating black bears (Ursus americanus). BMC Genomics 12, 171, doi:10.1186/1471-2164-12-171 (2011).
9 Fedorov, V. B. et al. Preservation of bone mass and structure in hibernating black bears (Ursus americanus) through elevated expression of anabolic genes. Funct Integr Genomics 12, 357-365, doi:10.1007/s10142-012-0266-3 (2012).
10 Fedorov, V. B. et al. Comparative functional genomics of adaptation to muscular disuse in hibernating mammals. Mol. Ecol. 23, 5524-5537, doi:10.1111/mec.12963 (2014).
11 Iles, T. L., Laske, T. G., Garshelis, D. L. & Iaizzo, P. A. Blood clotting behavior is innately modulated in Ursus americanus during early and late denning relative to summer months. J Exp. Biol. 220, 455-459, doi:10.1242/jeb.141549 (2017).
12 Nourse, J., Braun, J., Lackner, K., Huttelmaier, S. & Danckwardt, S. Large-scale identification of functional microRNA targeting reveals cooperative regulation of the hemostatic system. J Thromb. Haemost. 16, 2233-2245, doi:10.1111/jth.14290 (2018).
13 Wang, X. et al. Diagnostic potential of plasma microRNA signatures in patients with deep-vein thrombosis. Thromb. Haemost. 116, 328-336, doi:10.1160/TH16-01-0071 (2016).
14 Teruel, R. et al. Potential role of miRNAs in developmental haemostasis. PLoS One 6, e17648, doi:10.1371/journal.pone.0017648 (2011).
15 Teruel-Montoya, R., Rosendaal, F. R. & Martinez, C. MicroRNAs in hemostasis. J. Thromb. Haemost. 13, 170-181, doi:10.1111/jth.12788 (2015).
16 Smith, T., Heger, A. & Sudbery, I. UMI-tools: modeling sequencing errors in Unique Molecular Identifiers to improve quantification accuracy. Genome Res. 27, 491-499, doi:10.1101/gr.209601.116 (2017).
17 Friedlander, M. R., Mackowiak, S. D., Li, N., Chen, W. & Rajewsky, N. miRDeep2 accurately identifies known and hundreds of novel microRNA genes in seven animal clades. Nucleic Acids Res. 40, 37-52, doi:10.1093/nar/gkr688 (2012).
18 Davis, M. P., van Dongen, S., Abreu-Goodger, C., Bartonicek, N. & Enright, A. J. Kraken: a set of tools for quality control and analysis of high-throughput sequence data. Methods 63, 41-49, doi:10.1016/j.ymeth.2013.06.027 (2013).
19 Love, M. I., Huber, W. & Anders, S. Moderated estimation of fold change and dispersion for RNA-seq data with DESeq2. Genome Biol. 15, 550, doi:10.1186/s13059-014-0550-8 (2014).
20 Srivastava, A. et al. Genome assembly and gene expression in the American black bear provides new insights into the renal response to hibernation. DNA Res., doi:10.1093/dnares/dsy036 (2018).
21 Li, B. & Dewey, C. N. RSEM: accurate transcript quantification from RNA-Seq data with or without a reference genome. BMC Bioinformatics 12, 323, doi:10.1186/1471-2105-12-323 (2011).
22 Arroyo, A. B. et al. microRNAs in the haemostatic system: More than witnesses of thromboembolic diseases? Thromb. Res. 166, 1-9, doi:10.1016/j.thromres.2018.03.021 (2018).
23 Jiang, Z. et al. Circulating microRNA expression and their target genes in deep vein thrombosis: A systematic review and bioinformatics analysis. Medicine (Baltimore) 96, e9330, doi:10.1097/MD.0000000000009330 (2017).
24 Starikova, I. et al. Differential expression of plasma miRNAs in patients with unprovoked venous thromboembolism and healthy control individuals. Thromb. Res. 136, 566-572, doi:10.1016/j.thromres.2015.07.005 (2015).
25 Jin, Q. Q. et al. Integrating microRNA and messenger RNA expression profiles in a rat model of deep vein thrombosis. Int. J. Mol. Med. 40, 1019-1028, doi:10.3892/ijmm.2017.3105 (2017).
26 Tay, J. et al. Circulating MicroRNA as Thrombosis Sentinels: Caveats and Considerations. Semin. Thromb. Hemost. 44, 206-215, doi:10.1055/s-0037-1606568 (2018).
27 Zuo, B. Z., J.; You, L.; Zhao, Y.; Yang, J.; Weng, Z.; Dai, L.; Wu, W.; Ruan, C.; Yang, H. Plasma microRNAs characterising patients with immune thrombocytopenic purpura. Thromb. Haemost. 117, 1420-1431, doi:10.1160/TH16-06-0481 (2017).
28 Dahiya, N. & Atreya, C. D. RAP1 Downregulation by miR-320c reduces Platelet Activation in Ex Vivo Storage. Microrna, doi:10.2174/2211536607666180521094532 (2018).
29 Wu, P. Y., Zhang, X. D., Zhu, J., Guo, X. Y. & Wang, J. F. Low expression of microRNA-146b-5p and microRNA-320d predicts poor outcome of large B-cell lymphoma treated with cyclophosphamide, doxorubicin, vincristine, and prednisone. Hum. Pathol. 45, 1664-1673, doi:10.1016/j.humpath.2014.04.002 (2014).
30 Qin, C. Z. et al. Downregulation of MicroRNA-320d predicts poor overall survival and promotes the growth and invasive abilities in glioma. Chem. Biol. Drug Des. 89, 806-814, doi:10.1111/cbdd.12906 (2017).
31 Wang, D., Cui, G., Hu, S. & Wang, D. W. Subtypes of SERPINC1 mutations and the thrombotic phenotype of inherited antithrombin deficient individuals in Chinese Han population. Blood Cells Mol. Dis. 62, 38-41, doi:10.1016/j.bcmd.2016.10.029 (2016).
32 Alhenc-Gelas, M. et al. Thrombotic risk according to SERPINC1 genotype in a large cohort of subjects with antithrombin inherited deficiency. Thromb. Haemost. 117, 1040-1051, doi:10.1160/TH16-08-0635 (2017).
33 Su, J. et al. A small deletion in SERPINC1 causes type I antithrombin deficiency by promoting endoplasmic reticulum stress. Oncotarget 7, 76882-76890, doi:10.18632/oncotarget.12349 (2016).
34 Wang, D., Tian, M., Cui, G. & Wang, D. W. Antithrombin deficiency and decreased protein C activity in a young man with venous thromboembolism: a case report. Front. Med. 12, 319-323, doi:10.1007/s11684-017-0553-4 (2018).
35 Bhakuni, T. et al. Identification of 2 Novel Polymorphisms and rs3138521 in 5′ Untranslated Region of SERPINC1 Gene in North Indian Population With Deep Vein Thrombosis. Clin. Appl. Thromb. Hemost. 23, 865-870, doi:10.1177/1076029616652725 (2017).
36 Mulder, R. et al. SERPINC1 gene mutations in antithrombin deficiency. Br. J. Haematol. 178, 279-285, doi:10.1111/bjh.14658 (2017).
37 Arinell, K. et al. Physical inactivity and platelet function in humans and brown bears: A comparative study. Platelets 29, 87-90, doi:10.1080/09537104.2017.1336530 (2018).
38 Buller, H. R. et al. Factor XI antisense oligonucleotide for prevention of venous thrombosis. The New England journal of medicine 372, 232-240, doi:10.1056/NEJMoa1405760 (2015).
39 Garcia, C. K., Goldstein, J. L., Pathak, R. K., Anderson, R. G. & Brown, M. S. Molecular characterization of a membrane transporter for lactate, pyruvate, and other monocarboxylates: implications for the Cori cycle. Cell 76, 865-873 (1994).
40 Shu, L., Wang, Z., Wang, Q., Wang, Y. & Zhang, X. Signature miRNAs in peripheral blood monocytes of patients with gastric or breast cancers. Open Biol 8, doi:10.1098/rsob.180051 (2018).
41 Yu, J., Feng, Y., Wang, Y. & An, R. Aryl hydrocarbon receptor enhances the expression of miR-150-5p to suppress in prostate cancer progression by regulating MAP3K12. Arch. Biochem. Biophys. 654, 47-54, doi:10.1016/j.abb.2018.07.010 (2018).
42 Zhu, C., Huang, Q. & Zhu, H. Melatonin Inhibits the Proliferation of Gastric Cancer Cells Through Regulating the miR-16-5p-Smad3 Pathway. DNA Cell Biol. 37, 244-252, doi:10.1089/dna.2017.4040 (2018).
43 Zhang, G. et al. LncRNA MT1JP functions as a ceRNA in regulating FBXW7 through competitively binding to miR-92a-3p in gastric cancer. Mol. Cancer 17, 87, doi:10.1186/s12943-018-0829-6 (2018).
44 Shen, X., Li, Y., Sun, G., Guo, D. & Bai, X. miR-181c-3p and -5p promotes high-glucose-induced dysfunction in human umbilical vein endothelial cells by regulating leukemia inhibitory factor. Int. J. Biol. Macromol. 115, 509-517, doi:10.1016/j.ijbiomac.2018.03.173 (2018).
45 Chen, L. P., Zhang, N. N., Ren, X. Q., He, J. & Li, Y. miR-103/miR-195/miR-15b Regulate SALL4 and Inhibit Proliferation and Migration in Glioma. Molecules 23, doi:10.3390/molecules23112938 (2018).
46 Alderman, C. Y., Y. The anti-melanoma activity and oncogenic targets of hsa-miR-15a-5p. RNA and Disease 3 (2016).
47 Xu, P., Wang, J., Sun, B. & Xiao, Z. Comprehensive analysis of miRNAs expression profiles revealed potential key miRNA/mRNAs regulating colorectal cancer stem cell self-renewal. Gene 656, 30-39, doi:10.1016/j.gene.2018.02.065 (2018).
48 Wu, Y., Huang, J., Xu, H. & Gong, Z. Over-expression of miR-15a-3p enhances the radiosensitivity of cervical cancer by targeting tumor protein D52. Biomed. Pharmacother. 105, 1325-1334, doi:10.1016/j.biopha.2018.06.033 (2018).
49 Sun, L.-N. et al. SIRT1 suppresses colorectal cancer metastasis by transcriptional repression of miR-15b-5p. Cancer Lett. 409, 104-115, doi:10.1016/j.canlet.2017.09.001 (2017).
50 Gao, Z. Q. et al. Long non-coding RNA GASS antagonizes the chemoresistance of pancreatic cancer cells through down-regulation of miR-181c-5p. Biomed. Pharmacother. 97, 809-817, doi:10.1016/j.biopha.2017.10.157 (2018).
51 Jayne, D. et al. Effect of Robotic-Assisted vs Conventional Laparoscopic Surgery on Risk of Conversion to Open Laparotomy Among Patients Undergoing Resection for Rectal Cancer: The ROLARR Randomized Clinical Trial. JAMA 318, 1569-1580, doi:10.1001/jama.2017.7219 (2017).
52 Young, G. J. & Cohen, B. B. The process and outcome of hospital care for Medicaid versus privately insured hospital patients. Inquiry 29, 366-371 (1992).
53 Yan, R. et al. MicroRNA-150-5p affects cell proliferation, apoptosis, and EMT by regulation of the BRAF(V600E) mutation in papillary thyroid cancer cells. J. Cell. Biochem. 119, 8763-8772, doi:10.1002/jcb.27108 (2018).
54 Shang, J. et al. miR-15a-5p suppresses inflammation and fibrosis of peritoneal mesothelial cells induced by peritoneal dialysis via targeting VEGFA. J Cell. Physiol., doi:10.1002/jcp.27660 (2018).
55 Lindner, S. E. et al. The miR-15 family reinforces the transition from proliferation to differentiation in pre-B cells. EMBO Rep 18, 1604-1617, doi:10.15252/embr.201643735 (2017).
56 Cun, J. & Yang, Q. Bioinformatics-based interaction analysis of miR-92a-3p and key genes in tamoxifen-resistant breast cancer cells. Biomed. Pharmacother. 107, 117-128, doi:10.1016/j.biopha.2018.07.158 (2018).
57 Cai, B. et al. MiR-16-5p targets SESN1 to regulate the p53 signaling pathway, affecting myoblast proliferation and apoptosis, and is involved in myoblast differentiation. Cell Death Dis. 9, 367, doi:10.1038/s41419-018-0403-6 (2018).
58 Lin, C. L. et al. Norcantharidin induces mitochondrial-dependent apoptosis through Mcl-1 inhibition in human prostate cancer cells. Biochim Biophys Acta Mol Cell Res 1864, 1867-1876, doi:10.1016/j.bbamcr.2017.07.015 (2017).
59 Zhao, C. L., Y.; Chen, G.; Wang, F.; Shen, Z.; Zhou, R. Overexpression of miR-15b-5p promotes gastric cancer metastasis by regulating PAQR3. Oncol. Rep. 38, 352-358, doi:10.3892/or.2017/5673 (2016).
60 Salomon, O. et al. Patients with severe factor XI deficiency have a reduced incidence of deep-vein thrombosis. Thromb. Haemost. 105, 269-273, doi:10.1160/TH10-05-0307 (2011).

1. A composition comprising a microribonucleic acid (miRNA) antisense inhibitor, said inhibitor comprising a plurality of nucleic acids conjugated by phosphorothioate linkers that hybridizes with an miRNA binding site.

2. The composition of claim 1, wherein at least one of said plurality of nucleic acids comprises a modification.

3. The composition of claim 2, wherein said modification is selected from the group consisting of a 2′-OMe modification, a locked nucleic acid (LNA) modification or a 2′-deoxy modification.

4. The composition of claim 1, wherein the 3′ end of said miRNA antisense inhibitor comprises a GalNAc conjugate.

5. The composition of claim 1, wherein said miRNA antisense inhibitor is a SERPINC1 miRNA antisense inhibitor selected from the group consisting of anti200bc, anti200a+141, anti429, anti18a and anti19b.

6. A method, comprising:
a) providing;
i) a patient exhibiting at least one symptom of a cardiovascular thrombosis and comprising a serpine family C member 1 gene (SERPINC1), said SERPINC1 gene having at least one micro-ribonucleic acid (miRNA) binding site;
ii) an inhibitory compound having affinity for said at least one miRNA binding site;
b) administering said inhibitory compound such that said at least one symptom is reduced.

7. The method of claim 6, wherein said at least one miRNA binding site is a SERPINC1 miRNA binding site selected from the group consisting of an miR-141-3p binding site, an miR-200a-3p binding site, an miR-200b-5p binding site, and an miR-200c-3p binding site.

8. The method of claim 6, wherein said inhibitory compound is a SERPINC1 miRNA antisense inhibitor selected from the group consisting of anti200bc, anti200a+141, anti429, anti18a and anti19b.

9. The method of claim 6, wherein said administering further comprises increasing expression of antithrombin messenger ribonucleic acid (mRNA) from said SERPINC1 gene.

10. The method of claim 9, wherein said increased antithrombin RNA increases translation of an antithrombin protein.

11. The method of claim 6, wherein said administering further comprises preventing development of said cardiovascular thrombosis.

12. The method of claim 6, wherein said administering further comprises treating a developed cardiovascular thrombosis.

13. The method of claim 6, wherein said inhibitory compound is an antisense nucleic acid sequence.

14. The method of claim 6, wherein said inhibitory compound is an antibody.

15. The method of claim 6, wherein said antibody is a polyclonal antibody.

16. The method of claim 6, wherein said antibody is a monoclonal antibody.

17. A method of manufacture of a microribonucleoc acid (mRNA) antisense inhibitor comprising;
a) providing;
i) an automated RNA synthesizer on a 1 μmol GalNAc (TEG)CPG support; and
ii) at least one modification compound selected from the group consisting of 2′-OMe and locked nucleic acid (LNA) phosphoramidites;
b) synthesizing a plurality of nucleic acids on said (TEG)CPG support with a standard RNA phosphoramidite synthesis cycle, comprising (i) detritylation, (ii) coupling, (iii) capping, and (iv) iodine oxidation to phosphate or sulfurization, to form an oligonucleotide having a sequence that hybridizes to a microRNA binding site;
c) coupling said modification compounds to said oligonucleotide with 2-cyanoethyl phosphoramidite to form an miRNA antisense inhibitor;
d) deprotecting and cleaving said miRNA antisense inhibitor from said GalNAc (TEG)CPG support.

18. The method of claim 17, further comprising purifying said miRNA antisense inhibitor by anion exchange HPLC.

19. The method of claim 17, further comprising desalting said purified miRNA antisense inhibitor by Sephadex G-25.

20. The method of claim 17, further comprises characterizing said purified/desalted miRNA antisense inhibitor by electrospray ionization mass spectrometry (ESI-MS) analysis.


SEQUENCE LISTING (7 sequences)

SEQ ID NO: 1 - DNA, 120 bp, Artificial Sequence
  Synthetic
ttagtctttttgtcttttatttcaggtcccggatccggtggtggtgcaaatcaaagaactgctcctcagtggatgttgcctttacttctaggcctgccgtggccatcttactgggcagca

SEQ ID NO: 2 - DNA, 120 bp, Artificial Sequence
  Synthetic
tgctgcccagtaagatggccacggcaggcctagaagtaaaggcaacatccactgaggagcagttctttgatttgcaccaccaccggatccgggacctgaaataaaagacaaaaagactaa

SEQ ID NO: 3 - DNA, 120 bp, Artificial Sequence
  Synthetic
ttggatagtgtctgatctctaatactgcctggtaatgatgacggcctagaaagcttatcgataccgtcgactagagctcgctgatcagcctcgactgtgccttctagttgccagccatct

SEQ ID NO: 4 - DNA, 120 bp, Artificial Sequence
  Synthetic
agatggctggcaactagaaggcacagtcgaggctgatcagcgagctctagtcgacggtatcgataagctttctaggccgtcatcattaccaggcagtattagagatcagacactatccaa

SEQ ID NO: 5 - DNA, 120 bp, Artificial Sequence
  Synthetic
gttgtttgcccctcccccgtgccttccttgaccctggaaggtgccactcccactgtcctttcctaataaaatgaggaaattgcatcgcattgtctgagtaggtgtcattctattctgggg

SEQ ID NO: 6 - DNA, 120 bp, Artificial Sequence
  Synthetic
ccccagaatagaatgacacctactcagacaatgcgatgcaatttcctcattttattaggaaaggacagtgggagtggcaccttccagggtcaaggaaggcacgggggaggggcaaacaac

SEQ ID NO: 7 - DNA, 21 bp, Artificial Sequence
  Synthetic
gcgtaacagactcatccacag